(19) United States
(12) Patent Application Publication
(10) Pub. No.: US 2025/0304601 A1
(43) Pub. Date: Oct. 2, 2025

(54) Processes for Preparing KRAS Inhibitors

(71) Applicant: Incyte Corporation, Wilmington, DE (US)

(72) Inventors: Zhongjiang Jia, Wilmington, DE (US); Michelle Kroc, Wilmington, DE (US); Minyan Li, Wilmington, DE (US); Yi Li, Wilmington, DE (US); Qiyan Lin, Wilmington, DE (US); Pingli Liu, Wilmington, DE (US); Timothy Martin, Wilmington, DE (US); Bardia Soltanzadeh, Wilmington, DE (US); Naijing Su, Wilmington, DE (US); Joseph Tassone, Wilmington, DE (US); Michael Xia, Wilmington, DE (US); Jiacheng Zhou, Wilmington, DE (US)

(21) Appl. No.: 19/085,474

(22) Filed: Mar. 20, 2025

Related U.S. Application Data
(60) Provisional application No. 63/568,880, filed on Mar. 22, 2024.
(60) Provisional application No. 63/704,904, filed on Oct. 8, 2024.

(51) Int. Cl. C07D 519/00; B01J 23/44

(52) U.S. Cl. CPC C07D 519/00; B01J 23/44

(57) ABSTRACT

This disclosure provides efficient and scalable processes for preparing a KRAS inhibitor.

### RELATED APPLICATIONS

This application is related to U.S. Provisional Application No. 63/568,880, filed Mar. 22, 2024, and U.S. Provisional Application No. 63/704,904, filed Oct. 8, 2024, the content of both of which is incorporated in its entirety.

### BACKGROUND

Ras proteins are part of the family of small GTPases that are activated by growth factors and various extracellular stimuli. The Ras family regulates intracellular signaling pathways responsible for growth, migration, survival, and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13(11), 828-51). Most RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al. Nat. Rev. Drug. Discov., 2014, 13(11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al. Nat. Rev. Drug. Discov., 2014, 13(11), 828-51). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al. Nat. Rev. Drug. Discov., 2014, 13(11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

Efficient and scalable synthetic routes are required to prepare KRAS inhibitors. The processes disclosed herein meet this need by providing a scalable synthetic route to prepare chiral KRAS inhibitors.

### SUMMARY

Provided herein are processes for preparing KRAS inhibitors such as compounds of Formula I:

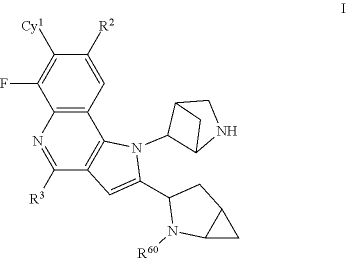

or pharmaceutically acceptable salts thereof, wherein the variables are as disclosed herein. Also disclosed are intermediates useful for preparing such KRAS inhibitors and processes of preparing such intermediates.

### DETAILED DESCRIPTION

Provided herein are processes for preparing potent and selective KRAS inhibitors and pharmaceutically acceptable salts thereof. An example of such a compound is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1*):

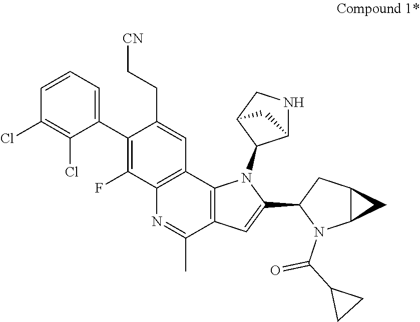 [SMILES: Cc1nc2c(F)c(-c3cccc(Cl)c3Cl)c(CCC#N)cc2c2c1cc([C@H]1C[C@H]3C[C@H]3N1C(=O)C1CC1)n2[C@H]1[C@H]2CN[C@@H]1C2]

or pharmaceutically acceptable salts thereof, including its atropisomer 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1), 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride salt (Compound 1-HCl), and its monochloride salt dihydrate (Compound-1-HCl·2H₂O). This compound is useful for the treatment of KRAS-mediated diseases, including a variety of cancers, as disclosed in PCT Application No. PCT/US2022/078048 (WO2023064857A1) and U.S. patent application Ser. No. 18/046,303 (US20230144051A1), the entire contents of which are incorporated herein by reference.

### I. Definitions

Listed below are definitions of various terms used to describe the processes provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein, and the laboratory procedures used in organic chemistry, and chemical manufacturing processes are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

The expressions “ambient temperature” and “room temperature” are understood in the art, and refer generally to a temperature, e.g., a reaction temperature, which is about the temperature of the room in which the reaction is carried out, e.g., a temperature from about 20° C. to about 30° C.

At various places in the present specification, variables defining divalent linking groups may be described. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “cycloalkyl,” employed alone or in combination with other terms, refers to a non-aromatic hydrocarbon ring system (monocyclic, bicyclic, or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

As used herein, the term “ester” refers to the replacement of the hydrogen of an acid by an alkyl group or another organic group. For example, esters are represented by —CO₂R, wherein R is a carbon atom of an organic group. Esters can also be in the form of boronic esters represented by —B(OR)₂, wherein R is a carbon atom of an organic group. Common boronic esters include allylboronic acid pinacol ester (also referred to as “pinacol ester”), phenyl boronic acid trimethylene glycol ester, and diisopropoxymethylborane.

Preparation of compounds provided herein can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4^{th} Ed., (Wiley, 2006).

As used herein, “protecting group” refers to a molecular framework that is introduced onto a specific functional group in a poly-functional molecule to block its reactivity under reaction conditions needed to make modifications elsewhere in the molecule. As such, the phrase “nitrogen protecting group” refers to a protecting group as described above that protects or masks a nitrogen atom. The phrase “hydrolysable protecting group” refers to a protecting group as described above that can be hydrolyzed or cleaved under acidic or basic conditions.

In some embodiments, the protecting group is benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), tert-butoxycarbonyl (Boc), 1-adamantyloxycarbonyl (Adoc), 2-adamantylcarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBoc), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, methoxymethyl (MOM), 2-methoxyethoxymethyl (MEM), t-butoxymethyl (Bum), benzyloxymethyl (BOM), or 2-tetrahydropyranyl (THP). In some embodiments, the protecting group is methoxymethyl (MOM), 2-methoxyethoxymethyl (MEM), allyl, t-butyldimethylsilyl (TBDMS or TBS), or pivoyl (Piv). In some embodiments, the protecting group is 2-(trimethylsilyl)ethoxymethyl (SEM), or tosyl (Ts). In some embodiments, the protecting group is tert-butoxycarbonyl (Boc).

As used herein, the phrase “protecting group reagent” refers to a reactant that installs a protecting group on another reactant in a process. Protecting group reagents include reactants that protect a free nitrogen atom or free oxygen atom. Examples of protecting group reagents include but are not limited to MOMCl, MEMCl, Boc₂O, TrtCl, SEMCl, BnCl, PivCl, TBDPSCl, TIPSCl, TMSCl, and BzCl.

As used herein, the term “coupling agent” refers to a chemical species that aids in the formation of a carbon-carbon bond in a reaction between a species having a leaving group and a reactive species. Exemplary coupling reagents include, but are not limited to, a palladium catalyst such as tetrakis(triphenylphosphine)palladium(0), bis(di-tert-butyl)-dimethylaminophenylphosphone dichloride palladium (II) (Pd-132), bis(triphenylphosphine)palladium(II) dichloride, and palladium (II) acetate in combination with reagents such as n-Bu₄NOAc, Cs₂CO₃, piperidine, copper iodide, diethylamine, K₂CO₃, NiCl₂-glyme, NiBr₂-glyme, potassium t-butoxide, potassium phosphate, and KOH.

As used herein, the term “halogenating agent” refers to a reagent that installs a halo group as defined supra on a reactant. As such, a brominating agent installs a bromo group on a reactant and an iodinating agent installs an iodo group on a reactant. Examples of halogenating agents include, but are not limited to, elemental halogens, e.g., chorine, bromine, or iodine, interhalogen compounds, e.g., BrF₃, IF₅, ICl, and N-haloimides, e.g., NCS, NBS, or NIS.

As used herein, the phrase “reducing agent” refers to a chemical species that donates an electron or hydride in a redox reaction. Examples of reducing agents include, but are not limited to, NaBH₄, LiAlH₄, sodium hydride, Red-AI, sodium amalgam, diborane, hydrogen gas, Xantphos, Cu(OAc)₂, and polymethylhydrosiloxane (PMHS), alone or in combination.

As used herein, the phrase “alkylating reagent” refers to chemical species that installs an alkyl group as defined supra on a reactant. Examples of alkylating agents include, but are not limited to, haloalkanes, such as bromoalkanes and iodoalkanes, e.g., Mel, and alkyl sulfonate esters, such as alkyl methanesulfonates, alkyl arenesulfonates, or alkyl trifluoromethanesulfoneates.

As used herein, the phrase “carbonylating agent” refers to chemical species that installs a carbonyl (C═O) group on a reactant. Examples of carbonylating agents include, but are not limited to, phosgene, triphosgene, and 1,1′-carbonyldimidazole.

The reactions of the processes described herein can be carried out at appropriate temperatures that can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

The reactions of the processes described herein can be carried out in air or under an inert atmosphere. Typically, reactions containing reagents or products that are substantially reactive with air can be carried out using air-sensitive synthetic techniques that are well known to the skilled artisan.

In some embodiments, preparation of compounds can involve the addition of acids or bases to effect, for example, catalysis of a desired reaction or formation of salt forms such as acid addition salts.

As used herein, the term “acid” refers to any species that can donate a proton or forming a covalent bond with an electron pair. Example acids can be inorganic or organic acids. Inorganic acids include HCl, hydrobromic acid, sulfuric acid, phosphoric acid, and nitric acid. Organic acids include formic acid, acetic acid, propionic acid, butanoic acid, benzoic acid, 4-nitrobenzoic acid, methanesulfonic acid, p-toluenesulfonic acid, benzenesulfonic acid, tartaric acid, trifluoroacetic acid, propiolic acid, butyric acid, 2-butynoic acid, vinyl acetic acid, pentanoic acid, hexanoic acid, heptanoic acid, octanoic acid, nonanoic acid and decanoic acid.

As used herein, the term “base” refers to any species that contains a filled orbital containing an electron pair which is not involved in bonding. Example bases include LiOH, NaOH, KOH, Li₂CO₃, Na₂CO₃, K₂CO₃, and Cs₂CO₃. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, i-propyl, n-butyl, t-butyl, TMS and cyclohexyl substituted amides.

The following abbreviations may be used herein: AcOH (acetic acid); aq. (aqueous); br (broad); calc. (calc.); Cs₂CO₃ (cesium carbonate); d (doublet); dd (doublet of doublets); DCM (dichloromethane); DIPEA (N,N-diisopropylethylamine); DIBAL (diisobutylaluminium hydride); DMF (N,N-dimethylformamide); DMSO (dimethyl sulfoxide); Et (ethyl); EtOAc (ethyl acetate); EtOH (ethanol); eq. (equivalent(s)); g (gram(s)); h (hour(s)); HCl (hydrochloric acid); HPLC (high performance liquid chromatography); Hz (hertz); J (coupling constant); K₂CO₃ (potassium carbonate); KOH (potassium hydroxide); L (liter(s)); LCMS (liquid chromatography-mass spectrometry); Li₂CO₃ (lithium carbonate); LiOH (lithium hydroxide); m (multiplet); M (molar); MS (mass spectrometry); Me (methyl); MeCN (acetonitrile); MeOH (methanol); mg (milligram(s)); min. (minutes(s)); mL (milliliter(s)); mmol (millimole(s)); mol (mole(s)); MTBE (methyl tert-butyl ether); N (normal); NaBH₄ (sodium borohydride); NaBH₃CN (sodium cyanoborohydride); Na₂CO₃ (sodium carbonate); NaHCO₃ (sodium bicarbonate); NaOH (sodium hydroxide); NBS (N-bromosuccinimide); NCS (N-chlorosuccinimide); NIS (N-iodosuccinimide); NEt₃ (triethylamine); NLT (not less than); nM (nanomolar); NMP (N-methyl-2-pyrrolidinone); NMR (nuclear magnetic resonance spectroscopy); OTf (trifluoromethanesulfonate); Ph (phenyl); pM (picomolar); r.t. (room temperature), s (singlet); t (triplet or tertiary); tert (tertiary); tt (triplet of triplets); TFA (trifluoroacetic acid); THF (tetrahydrofuran); TMS (trimethylsilyl) wt % (weight percent). Brine is sat. aq. sodium chloride. In vacuo is under vacuum.

Compounds of the present disclosure can exist in the form of atropisomers (i.e., conformational diastereoisomers) that can be stable at ambient temperature and separable, e.g., by chromatography. For example, compounds provided herein can exist in the form of atropisomers in which the conformation of the dichlorophenyl relative to the remainder of the molecule is as shown by the partial formulae Formula (II-A) or Formula (II-B) below. Reference to the compounds described herein or any of the embodiments is understood to include all such atropisomeric forms of the compounds, including, without limitation, the atropisomeric forms represented by Formula (II-A) or Formula (II-B) below. The asymmetry of atropisomers is assigned as either Rₐ or Sₐ, as determined by conventional methods of characterizing points of asymmetry.

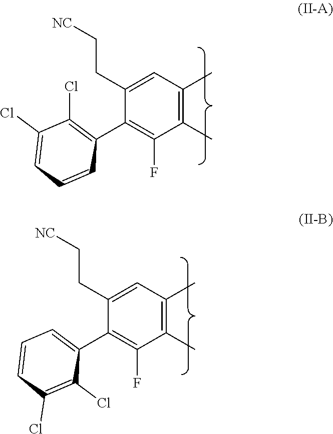

For example, Compound 1* can exist as two atropisomers that are stable at ambient temperature, 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1) and 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

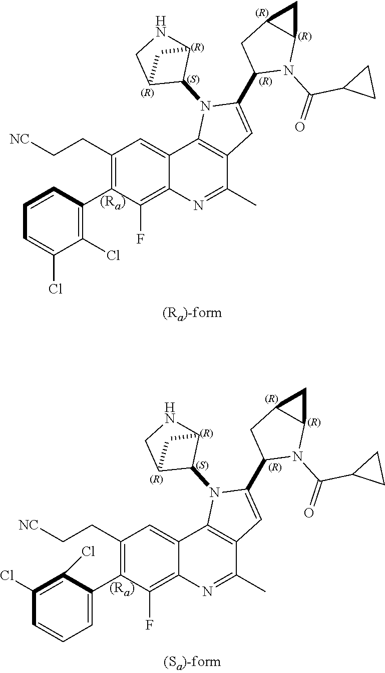

An aspect of the present disclosure relates to methods for the preparation of compounds of Formula I, including Compound 1*, and intermediates used for the preparation of compounds of Formula I, including Compound 1*, wherein one atropisomer is in excess compared to the other isomer. The term “atropisomeric purity” refers to the percentage of a given atropisomer present in the compound compared to the total amount of the compound. In some embodiments, the atropisomeric purity of each of the atropisomeric compounds described herein can be greater than 50%, such as about 80% or greater, about 90% or greater, about 95% or greater, about 96% or greater, about 97% or greater, about 98% or greater, about 99% or greater, about 99.5% or greater, or about 99.9% or greater. When the enantiomeric purity of an atropisomeric compound is about 95% or greater, about 96% or greater, about 97% or greater, about 98% or greater, about 99% or greater, about 99.5% or greater, or about 99.9% or greater, the atropisomeric compound can be referred to as being “substantially free” of the alternative atropisomer.

### II. Processes

In an aspect, provided herein is a process for preparing a compound of Formula I:

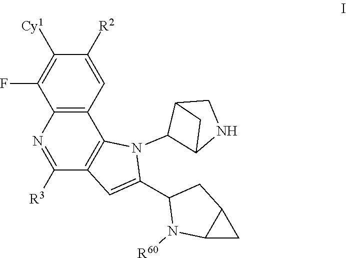

wherein
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
comprising deprotecting a compound of Formula II:

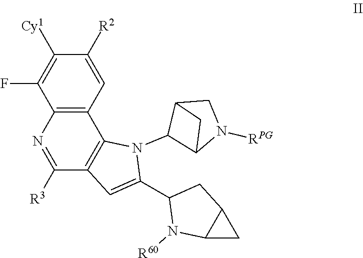

wherein
R^{PG} is a nitrogen protecting group;
to produce the compound of Formula I.

In some embodiments, R² is CH₂CH₂CN.

In some embodiments, Cy¹ is 2,3-dichlorophenyl.

In some embodiments, R³ is methyl.

In some embodiments, R⁶⁰ is C(O)R^{b60}. In some embodiments, R^{b60} is cyclopropyl.

In some embodiments, R^{PG} is a hydrolysable protecting group and the deprotecting comprises hydrolyzing the compound of Formula II. In some embodiments of the processes herein, R^{PG} is tert-butyloxycarbonyl. In other embodiments, the deprotecting comprises reacting the compound of Formula II with an acid. In some embodiments, the deprotecting comprises reacting the compound of Formula II with a Lewis acid. In yet other embodiments, the acid is HCl. In other embodiments, the acid is a trialkylsilyl halide, for example TMSI (trimethylsilyl iodide).

In some embodiments, the reaction can be carried out in the presence of a hydroxylic solvent or mixtures thereof, e.g., water, MeOH or EtOH. In some embodiments, the reaction can be carried out in the presence of a halogenated solvent or mixtures thereof, e.g., DCM, chloroform, 1,2-dichloroethane, or 1,1,1-trichloroethane. In some embodiments, the reaction can be carried out in more than one stage, e.g., treatment with a Lewis acid in the presence of a halogenated solvent, followed by addition of a hydroxylic solvent.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at r.t.

In another aspect, provided herein is a process for preparing a compound of Formula II:

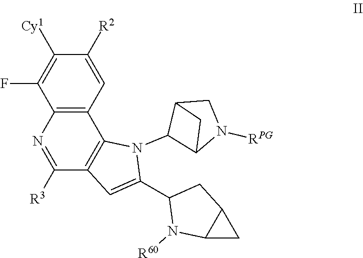

wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
comprising cyclizing a compound of Formula III:

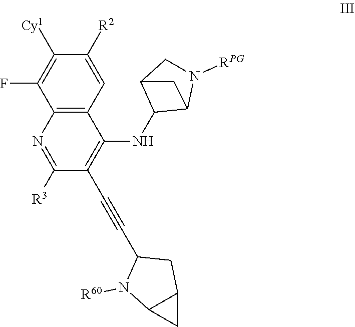

to form the compound of Formula II.

In some embodiments, R² is CH₂CH₂CN.

In some embodiments, Cy¹ is 2,3-dichlorophenyl.

In some embodiments, R³ is methyl.

In some embodiments, R⁶⁰ is C(O)R^{b60}. In some embodiments, R^{b60} is cyclopropyl.

In some embodiments of the processes herein, R^{PG} is tert-butyloxycarbonyl.

In some embodiments, the cyclizing comprises reacting the compound of Formula III with a base. Examples of suitable bases include alkali metal carbonates, e.g., K₂CO₃ or Cs₂CO₃.

In some embodiments, the reaction can be carried out in the presence of a polar aprotic solvent or mixtures thereof, e.g., THF, 1,4-dioxane, MeCN, DMF, DMSO or NMP.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., about 150° C. In some embodiments, the reaction can be carried out at a temperature from about 80° C. to about 90° C., or from about 80° C. to about 85° C.

In yet another aspect, provided herein is a process of preparing a compound of Formula III:

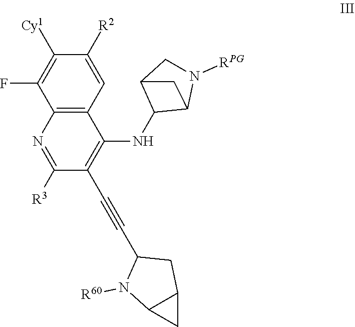

comprising
coupling a compound of Formula IV:

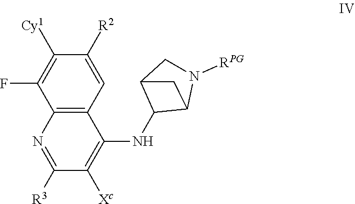

with a compound of Formula V:

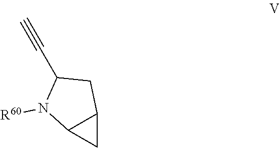

to form the compound of Formula III
wherein
X^{c} is Cl, Br, or I;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl.

In some embodiments, X^{c} is Cl. In some embodiments, X^{c} is Br. In some embodiments, X^{c} is I.

In some embodiments, R² is CH₂CH₂CN.

In some embodiments, Cy¹ is 2,3-dichlorophenyl.

In some embodiments, R³ is methyl.

In some embodiments, R⁶⁰ is C(O)R^{b60}. In some embodiments, R^{b60} is cyclopropyl.

In some embodiments, R^{PG} is tert-butyloxycarbonyl.

In some embodiments, X^{c} is I.

The coupling can be carried out in the presence of a palladium catalyst. Suitable palladium catalysts can include palladium(0) catalysts, e.g., Pd₂(dba)₃ or Pd(PPh₃)₄ and palladium(II) catalysts, e.g., Pd(PPh₃)₂Cl₂, Pd(dppe)Cl₂, Pd(dppp)Cl₂, or Pd(dppf)Cl₂. Palladium(II) catalysts also include, e.g., palladium (II) acetate. In some embodiments, the reaction can optionally be carried out in the presence of a cocatalyst, such as a copper cocatalyst, e.g., a copper halide, such as CuI, but the reaction can also be carried out under copper-free conditions. The coupling can be carried out in the presence of a base. Suitable bases can include amine bases, e.g., NEt₃ or DIPEA. Other suitable bases include alkali metal carbonates, e.g., K₂CO₃ or Cs₂CO₃. Other suitable bases include carboxylic acid salts such as acetate salts, e.g., NaOAc or n-Bu₄NOAc.

The coupling can be carried out in the presence of a phosphine reagent. Suitable phosphine reagents can include tris (4-fluorophenyl)phosphine and triphenylphosphine.

In some embodiments, the reaction can be carried out in the presence of a polar aprotic solvent or mixtures thereof, e.g., THF, 1,4-dioxane, MeCN, DMF, or NMP. Polar, aprotic solvents also include, e.g., DMSO.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., about 100° C. In some embodiments, the reaction can be carried out at a temperature from about 60° C. to about 80° C., such as about 80° C.

In still another aspect, provided herein is a process of preparing a compound of Formula IV:

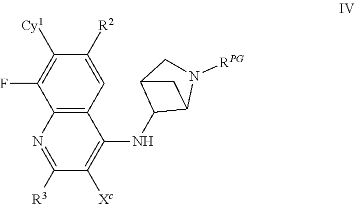

wherein
X^{c} is Cl, Br, or I;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
comprising halogenating a compound of Formula VI:

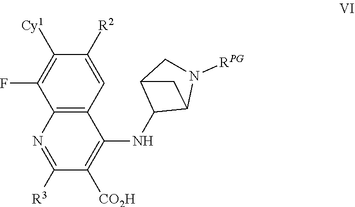

to form the compound of Formula IV.

In some embodiments, R² is CH₂CH₂CN.

In some embodiments, Cy¹ is 2,3-dichlorophenyl.

In some embodiments, R³ is methyl.

In some embodiments of the processes herein, R^{PG} is tert-butyloxycarbonyl.

In some embodiments, X^{c} is Cl. In some embodiments, X^{c} is Br. In some embodiments, X^{c} is I.

In some embodiments, the halogenating comprises reacting the compound of Formula IV with a halogenating agent.

In some embodiments, the halogenating is chlorinating, and the halogenating agent is a chlorinating agent, e.g., NCS. In some embodiments the halogenating is brominating, and the halogenating agent is a brominating agent, e.g., NBS. In some embodiments, the halogenating is iodinating, and the halogenating agent is an iodinating agent, e.g., NIS.

In some embodiments, the halogenating comprises reacting the compound of Formula IV with an iodinating agent. An example of a suitable iodinating agent is N-iodosuccinimide.

The halogenating can be carried out in the presence of a base. In still other embodiments, the base is a phosphate base. In some embodiments, the base is trisodium phosphate.

In some embodiments, the reaction can be carried out in the presence of a polar aprotic solvent or mixtures thereof, e.g., THF, 1,4-dioxane, or MeCN.

In some embodiments, the reaction can be carried out at a temperature in the range from about 0° C. to about 50° C., such as a temperature of about 5° C., about 10° C., about 15° C., about 20° C., about 25° C., about 30° C., about 40° C., or about 50° C. In some embodiments, the reaction can be carried out at a temperature from about 20° C. to about 30° C. In some embodiments, the reaction can be carried out at about r.t.

In an aspect, provided herein is a process of preparing a compound of Formula VI:

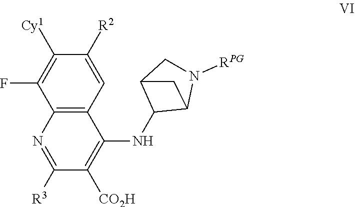

comprising hydrolyzing a compound of Formula VII:

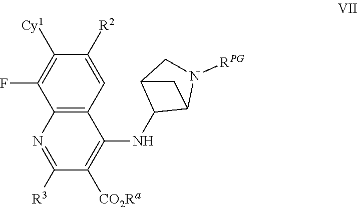

wherein
R^{a} is C₁₋₃ alkyl;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
to form the compound of Formula VI.

In some embodiments, R² is CH₂CH₂CN.

In some embodiments, Cy¹ is 2,3-dichlorophenyl.

In some embodiments, R³ is methyl.

In some embodiments of the processes herein, R^{PG} is tert-butyloxycarbonyl.

In some embodiments, R^{a} is ethyl.

In some embodiments, the hydrolyzing is carried out by reacting the compound of Formula VI in the presence of a base. Examples of suitable bases include alkali metal carbonate bases such as K₂CO₃ or Cs₂CO₃. Other examples of suitable bases include alkali or alkaline earth hydroxide bases such as NaOH or KOH. Further examples of suitable bases include alkali metal trialkylsiloxide bases such as NaOTMS or KOTMS. In other embodiments, the base is NaOTMS. In yet other embodiments, the base is NaOH.

In some embodiments, the reaction can be carried out in the presence of a hydroxylic solvent or mixtures thereof, e.g., water, MeOH or EtOH. In some embodiments, the reaction can be carried out in the presence of water. In some embodiments, the reaction can be carried out in the presence of water and one or more water-miscible co-solvents, e.g., THF, 1,4-dioxane, MeCN, MeOH or EtOH.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., about 100° C. In some embodiments, the reaction can be carried out at a temperature from about 40° C. to about 60° C., such as about 50° C.

In another aspect, provided herein is a process of preparing a compound of Formula VII:

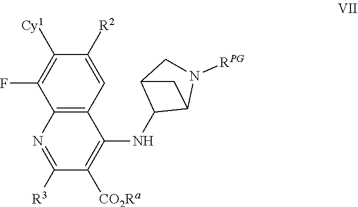

wherein
R^{a} is C₁₋₃ alkyl;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
comprising reacting a compound of Formula VIII:

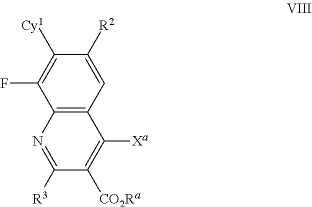

wherein
X^{a} is halo or OH;
with a compound of Formula IX:

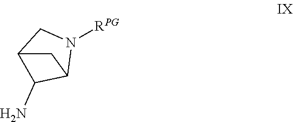

wherein
R^{PG} is a nitrogen protecting group;
to form the compound of Formula VII.

In some embodiments, R² is CH₂CH₂CN.

In some embodiments, Cy¹ is 2,3-dichlorophenyl.

In some embodiments, R³ is methyl.

In some embodiments, R^{PG} is tert-butyloxycarbonyl.

In some embodiments, R^{a} is ethyl.

In some embodiments, X^{a} is Cl or OH. In some embodiments, X^{a} is Cl. In some embodiments, X^{a} is OH.

In some embodiments, the reacting is performed in the presence of a base. In other embodiments, the base is a trialkylamine base such as NEt₃ or DIPEA. In some embodiments, the reacting is performed in the absence of a base.

In some embodiments, the compound of Formula IX is a salt thereof. In some embodiments, the compound of Formula IX is an oxalate salt.

In some embodiments, the reaction is carried out in the presence of a lithium salt, e.g., LiCl or Li₂CO₃.

In some embodiments, the reaction can be carried out in the presence of a polar aprotic solvent or mixtures thereof, e.g., THF, 1,4-dioxane, MeCN, DMF, DMSO, or NMP.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., about 150° C. In some embodiments, the reaction can be carried out at a temperature from about 70° C. to about 90° C., or at about 80° C.

In yet another aspect, provided herein is a process of preparing a compound of Formula VIII:

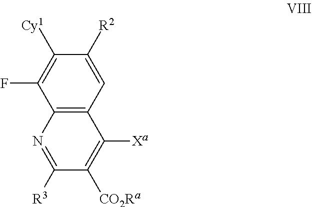

wherein
R^{a} is C₁₋₃ alkyl;
X^{a} is halo or OH;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
comprising reducing a compound of Formula X:

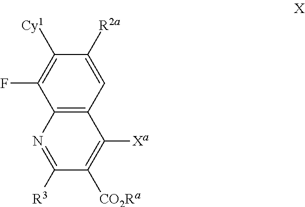

wherein
R^{2a} is selected from C₂₋₄ alkenyl optionally substituted with CN;
to form the compound of Formula VIII.

In some embodiments, R^{2a} is CH═CHCN and R² is CH₂CH₂CN.

In some embodiments, Cy¹ is 2,3-dichlorophenyl.

In some embodiments, R³ is methyl.

In some embodiments, R^{a} is ethyl.

In some embodiments, X^{a} is Cl or OH. In some embodiments, X^{a} is Cl. In some embodiments, X^{a} is OH.

In some embodiments, the reducing comprises reacting the compound of Formula X with a reducing agent. The reducing agent can be a silane reducing agent such as polymethylhydrosiloxane (PMHS). The reaction can be carried out in the presence of a copper catalyst formed by a suitable copper (II) salt such as Cu(OAc)₂ and a suitable ligand such as Xantphos or DPEphos. In other embodiments the reducing agent can be a borohydride such as NaBH₄ or NaBH₃CN.

In some embodiments, the reaction can be carried out in the presence of suitable solvent or mixtures thereof, e.g., toluene, tert-butanol, THF, 1,4-dioxane, MeCN or pyridine.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 40° C. to about 70° C., from about 50° C. to about 60° C. or at about 50° C. or about 60° C.

In still another aspect, provided herein is a process of preparing a compound of Formula X-A:

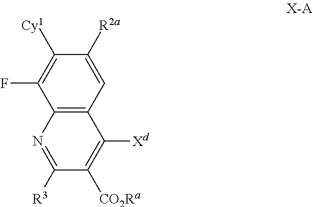

wherein
R^{a} is C₁₋₃ alkyl;
X^{d} is halo;
R^{2a} is selected from C₂₋₄ alkyl and C₂₋₄ alkenyl, both of which are optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
comprising halodehydroxylating a compound of Formula XI:

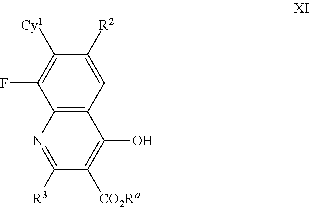

to form the compound of Formula X.

In some embodiments, R² is CH₂CH₂CN. In some embodiments, R² is CH═CHCN.

In some embodiments, R^{2a} is CH═CHCN.

In some embodiments, Cy¹ is 2,3-dichlorophenyl.

In some embodiments, R³ is methyl.

In some embodiments, R^{a} is ethyl.

In some embodiments, X^{d} is Cl, Br, or I. In some embodiments, X^{d} is Cl.

In some embodiments, the halodehydroxylating comprises chlorodehydroxylating. The halodehydroxylating can be performed in the presence of suitable halodehydroxylating (or chlorodehydroxylating agent) such as phosphoryl chloride (POCl₃) or thionyl chloride. In yet other embodiments, the halodehydroxylating is performed in the presence of a suitable catalyst such as benzyltriethylammonium chloride (BTEAC).

In some embodiments, the reaction can be carried out in the presence of suitable solvent or mixtures thereof, e.g., toluene, THF, 1,4-dioxane, N,N-diethylaniline, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 10° C. to about 70° C., such as from about 20° C. to about 60° C.

In an aspect, provided herein is a process of preparing a compound of Formula XI:

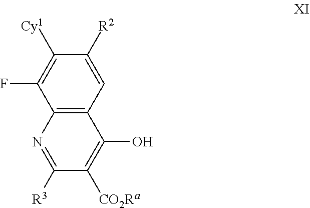

wherein
R^{a} is C₁₋₃ alkyl;
Cy¹ is phenyl; wherein the phenyl is optionally substituted with 1 or 2 substituents independently selected from R¹⁰;
R² is selected from C₂₋₄ alkenyl optionally substituted with CN;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
comprising coupling a compound of Formula XII:

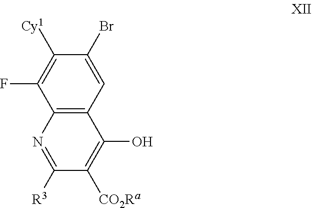

with a C₂₋₄ alkene that is optionally substituted with CN to form the compound of Formula XI.

In some embodiments, R² is CH═CHCN.

In some embodiments, Cy¹ is 2,3-dichlorophenyl.

In some embodiments, R³ is methyl.

In some embodiments, R^{a} is ethyl.

In some embodiments, the C₂₋₄ alkene that is optionally substituted with CN is acrylonitrile.

The coupling can be performed in the presence of a catalyst. The catalyst can be a palladium catalyst, such as a palladium(0) or palladium(II) catalyst. In yet other embodiments, the palladium catalyst is bis(di-tert-butyl)-(4-dimethylaminophenyl)phosphine)dichloridopalladium (II) (Pd-132).

In some embodiments, the reaction can be carried out in the presence of suitable solvent or mixtures thereof, e.g., toluene, THF, 1,4-dioxane, N,N-diethylaniline, or MeCN.

In some embodiments, the reaction can be carried out at a temperature in the range from about 0° C. to about 120° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., or about 120° C. In some embodiments, the reaction can be carried out at a temperature from about 70° C. to about 100° C., or from about 80° C. to about 90° C., such as at about 85° C.

In another aspect, provided herein is a process of preparing a compound of Formula XII:

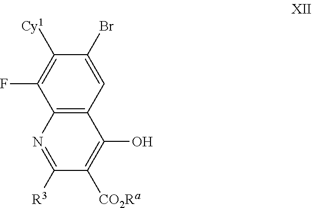

wherein
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
comprising reacting a compound of Formula XIII:

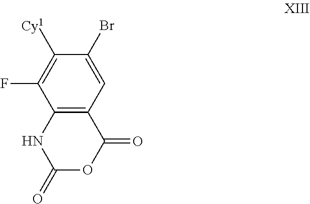

with a compound of Formula XIV:

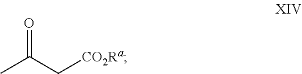

wherein R^{a} is C₁₋₃ alkyl;
   to form the compound of Formula XII.

In some embodiments, Cy¹ is 2,3-dichlorophenyl.

In some embodiments, R³ is methyl.

In some embodiments, R^{a} is ethyl.

In some embodiments, the reacting is carried out in the presence of a base. Suitable bases include carboxylic acid salts, such as acetate salts. Example of suitable carboxylic acid salts include ammonium, tetraalkylammonium or alkali metal salts. In some embodiments the base is an alkali metal acetate salts such as sodium acetate or potassium acetate.

In some embodiments, the reaction can be carried out in the presence of suitable solvent or mixtures thereof, e.g., toluene, xylene, or DMSO.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., or about 150° C. In some embodiments, the reaction can be carried out at a temperature from about 50° C. to about 120° C., or from about 50° C. to about 100° C.

In another aspect, provided herein is a process of preparing a compound of Formula XII:

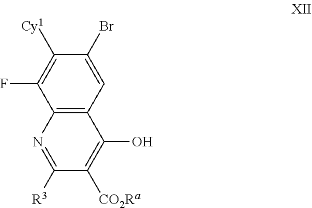

wherein
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
comprising reacting a compound of Formula XIII:

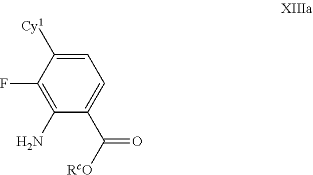

wherein R^{c} is C₁₋₃ alkyl;
   with a compound of Formula XIV:

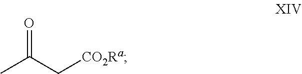

wherein R^{a} is C₁₋₃ alkyl;
   to form the compound of Formula XII.

In some embodiments, Cy¹ is 2,3-dichlorophenyl.

In some embodiments, R³ is methyl.

In some embodiments, R^{a} is ethyl.

In some embodiments, R^{c} is methyl.

In some embodiments, the reacting is carried out in the presence of a base. Suitable bases include metal alkoxides. Example of suitable alkoxides include methoxide and ethoxide. In some embodiments the base is metal alkoxide such as sodium ethoxide, sodium methoxide, and potassium methoxide.

In some embodiments, the reaction can be carried out in the presence of suitable solvent or mixtures thereof, e.g., toluene, xylene or DMSO.

In some embodiments, the reaction can be carried out at a temperature in the range from about 0° C. to about 150° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., about 120° C., about 130° C., about 140° C., or about 150° C. In some embodiments, the reaction can be carried out at a temperature from about 50° C. to about 120° C., or from about 50° C. to about 100° C. In some embodiments, the reaction can be carried out at reflux.

In yet another aspect, provided herein is a process of preparing a compound of Formula XIII:

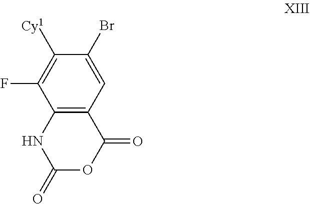

wherein
Cy¹ is phenyl; wherein the phenyl is optionally substituted with 1 or 2 substituents independently selected from R¹⁰; and
each R¹⁰ is independently selected from C₁₋₃ alkyl and halo;
comprising carbonylating a compound of Formula XV:

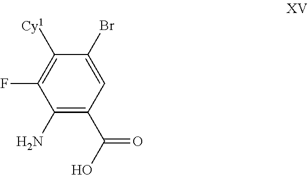

to form the compound of Formula XIII.

In some embodiments, Cy¹ is 2,3-dichlorophenyl.

In some embodiments, the carbonylating comprises reacting the compound of Formula XV with a carbonylating agent such as triphosgene.

In some embodiments, the reaction can be carried out in the presence of suitable solvent or mixtures thereof. Suitable solvents can include polar aprotic solvents and mixtures thereof, e.g., THF, 1,4-dioxane, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 50° C. to about 70° C., such as at about 60° C.

In still another aspect, provided herein is a process of preparing a compound of Formula XV:

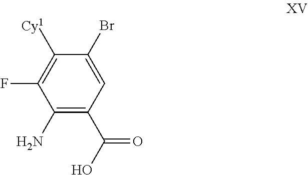

wherein
Cy¹ is phenyl; wherein the phenyl is optionally substituted with 1 or 2 substituents independently selected from R¹⁰;
each R¹⁰ is independently selected from C₁₋₃ alkyl and halo; and
comprising hydrolyzing a compound of Formula XVI:

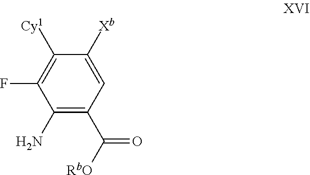

wherein
X^{b} is Cl, Br, or I;
R^{b} is C₁₋₃ alkyl;
   to form the compound of Formula XV.

In some embodiments, Cy¹ is 2,3-dichlorophenyl.

In some embodiments, R^{b} is methyl.

In some embodiments, X^{b} is Br.

In some embodiments, the hydrolyzing comprises reacting the compound of Formula XVI in the presence of a base. Examples of suitable bases include alkali metal carbonate bases such as K₂CO₃ or Cs₂CO₃. Other examples of suitable bases include alkali or alkaline earth hydroxide bases such as NaOH or KOH. Further examples of suitable bases include alkali metal trialkylsiloxide bases such as NaOTMS or KOTMS. In other embodiments, the base is NaOTMS. In yet other embodiments, the base is NaOH.

In some embodiments, the reaction can be carried out in the presence of a hydroxylic solvent or mixtures thereof, e.g., water, MeOH or EtOH. In some embodiments, the reaction can be carried out in the presence of water. In some embodiments, the reaction can be carried out in the presence of water and one or more water-miscible co-solvents, e.g., THF, 1,4-dioxane, MeCN, MeOH or EtOH.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., about 100° C. In some embodiments, the reaction can be carried out at a temperature from about 40° C. to about 60° C., such as about 50° C.

In an aspect, provided herein is a process of preparing a compound of Formula XVI:

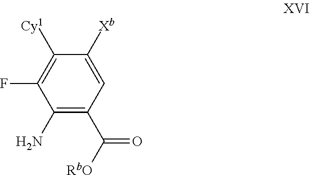

wherein
X^{b} is Cl, Br, or I;
Cy¹ is phenyl; wherein the phenyl is optionally substituted with 1 or 2 substituents independently selected from R¹⁰;
each R¹⁰ is independently selected from C₁₋₃ alkyl and halo; and
R^{b} is C₁₋₃ alkyl;
comprising halogenating a compound of Formula XVII:

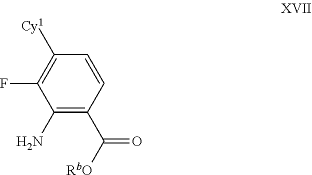

to form the compound of Formula XVI.

In some embodiments, Cy¹ is 2,3-dichlorophenyl.

In some embodiments, R^{b} is methyl.

In some embodiments, X^{b} is Br.

In some embodiments, the halogenating comprises reacting the compound of Formula VII with a halogenating agent. In other embodiments, the halogenating is brominating. In yet other embodiments, the halogenating agent is a brominating agent. In still other embodiments, the halogenating agent is N-bromosuccinimide (NBS).

In some embodiments, the reaction can be carried out in the presence of suitable solvent or mixtures thereof. Suitable solvents can include polar aprotic solvents and mixtures thereof, e.g., THF, 1,4-dioxane, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 40° C. to about 80° C., or from about 50° C. to about 70° C. such as at about 60° C.

In another aspect, provided herein is a process of preparing a compound of Formula XVII:

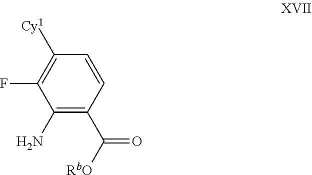

wherein
R^{b} is C₁₋₃ alkyl;
Cy¹ is phenyl; wherein the phenyl is optionally substituted with 1 or 2 substituents independently selected from R¹⁰;
comprising coupling a compound of Formula XVIII:

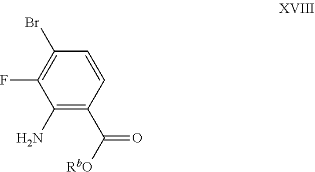

with a compound of Formula XIX:

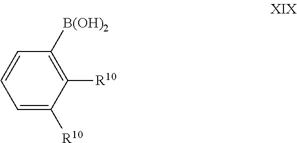

or an ester thereof;
   wherein
each R¹⁰ is independently selected from C₁₋₃ alkyl and halo;
   in the presence of a palladium catalyst to form the compound of Formula XVII.

In some embodiments, Cy¹ is 2,3-dichlorophenyl.

In some embodiments, R^{b} is methyl.

In some embodiments, each R¹⁰ is Cl.

In some embodiments, the coupling is performed in the presence of a catalyst. The catalyst can a palladium catalyst, such as a palladium(0) or palladium(II) catalyst. In yet other embodiments, the palladium catalyst is bis(di-tert-butyl(4-dimethylaminophenyl)phosphine)dichloropalladium(II) (Pd-132).

In some embodiments, the reaction can be carried out in the presence of a suitable base. In some embodiments, the base is an alkali metal carbonate base e.g., K₂CO₃ or Cs₂CO₃. In some embodiments, the base is an alkali metal fluoride base, e.g., KF or CsF.

In some embodiments, the reaction can be carried out in the presence of suitable solvent or mixtures thereof, e.g., THF, 1,4-dioxane, or MeCN.

In some embodiments, the reaction can be carried out at a temperature in the range from about 0° C. to about 120° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., or about 120° C. In some embodiments, the reaction can be carried out at a temperature from about 50° C. to about 90° C., or from about 60° C. to about 80° C., such as at about 70° C.

In yet another aspect, provided herein is a process of preparing a compound of Formula XVIII:

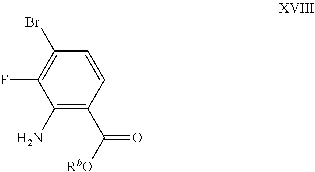

wherein
R^{b} is C₁₋₃ alkyl;
comprising esterifying a compound of Formula XX:

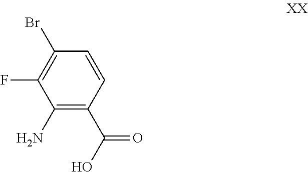 [SMILES: Nc1c(C(=O)O)ccc(Br)c1F]

to form the compound of Formula XVIII.

In some embodiments, R^{b} is methyl.

In some embodiments, the esterifying comprises reacting the compound of Formula XX with an alkylating agent. In some embodiments, the alkylating agent is (R^{b})₂SO₄, wherein R^{b} is C₁₋₃ alkyl. In yet other embodiments, the alkylating agent is dimethyl sulfate.

In still other embodiments, the reacting is performed in the presence of a base. In some embodiments, the base is an alkali metal carbonate base e.g., K₂CO₃ or Cs₂CO₃.

In some embodiments, the reaction can be carried out in the presence of suitable solvent or mixtures thereof, e.g., THF, 1,4-dioxane, or MeCN.

In some embodiments, the reaction can be carried out at a temperature in the range from about 0° C. to about 100° C., such as a temperature of about 5° C., about 10° C., about 15° C., about 20° C., about 25° C., about 30° C., about 40° C., about 50° C., about 60° C., about 70° C., about 80° C., about 90° C., or about 100° C. In some embodiments, the reaction can be carried out at a temperature from about 0° C. to about 50° C., or from about 5° C. to about 50° C., such as at about 20° C.

In some embodiments of the processes herein, R² is CH₂CH₂CN.

In some embodiments of the processes herein, R² is CH═CHCN.

In some embodiments of the processes herein, R^{2a} is CH═CHCN.

In some embodiments of the processes herein, Cy¹ is 2,3-dichlorophenyl.

In some embodiments of the processes herein, R³ is methyl or ethyl. In some embodiments, R³ is methyl.

In some embodiments of the processes herein, R⁶⁰ is C(O)R^{b60}. In some embodiments, R^{b60} is cyclopropyl.

In some embodiments of the processes herein, R^{PG} is tert-butyloxycarbonyl.

In some embodiments of the processes herein, X is 1.

In some embodiments of the processes herein, X^{a} is Cl or OH. In some embodiments of the processes herein, X^{a} is Cl. In some embodiments of the processes herein, X^{a} is OH.

In some embodiments of the processes herein, X^{b} is Br.

In some embodiments of the processes herein, X^{c} is 1.

In some embodiments of the processes herein, R^{a} is methyl or ethyl. In some embodiments, R^{a} is ethyl.

In some embodiments of the processes herein, R^{b} is methyl or ethyl. In some embodiments, R^{b} is methyl.

In some embodiments of the processes herein, R^{c} is methyl or ethyl. In some embodiments, R^{c} is methyl.

In some embodiments of the processes herein, the compound of Formula I is 3-(1-(2-azabicyclo[2.1.1]hexan-5-yl)-2-(2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt, hydrate, of solvate thereof. In still other embodiments, the compound of Formula I is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1*), or a pharmaceutically acceptable salt thereof. In some embodiments, the compound of Formula I is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1). In some embodiments, the compound of Formula I is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride salt (Compound 1-HCl). In some embodiments, the compound of Formula I is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride salt dihydrate (Compound 1-HCl·2H₂O). In other embodiments, the compound of Formula I is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

In some embodiments of the processes herein, the compound of Formula II is tert-butyl 5-(8-(2-cyanoethyl)-2-(2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate. In still other embodiments, the compound of Formula II is tert-butyl (1R,4R,5S)-5-(8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (Compound 2*). In some embodiments, the compound of Formula II is tert-butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (Compound 2). In other embodiments, the compound of Formula II is tert-butyl (1R,4R,5S)-5-((Sₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

In some embodiments of the processes herein, the compound of Formula III is tert-butyl 5-((6-(2-cyanoethyl)-3-((2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate. In still other embodiments, the compound of Formula III is tert-butyl (1R,4R,5S)-5-((6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate (Compound 3*). In some embodiments, the compound of Formula III is tert-butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate (Compound 3). In other embodiments, the compound of Formula III is tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate.

In some embodiments of the processes herein, the compound of Formula IV is tert-butyl 5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate. In still other embodiments, the compound of Formula IV is tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (Compound 4*). In some embodiments, the compound of Formula IV is tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (Compound 4). In other embodiments, the compound of Formula IV is tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

In some embodiments of the processes herein, the compound of Formula V is cyclopropyl(3-ethynyl-2-azabicyclo[3.1.0]hexan-2-yl)methanone. In some embodiments of the processes herein, the compound of Formula V is cyclopropyl((1R,3R,5R)-3-ethynyl-2-azabicyclo[3.1.0]hexan-2-yl)methanone (Compound 5).

In some embodiments of the processes herein, the compound of Formula VI is 4-((2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid. In still other embodiments, the compound of Formula VI is 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (Compound 6*). In some embodiments of the processes herein, the compound of Formula VI is (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (Compound 6). In other embodiments, the compound of Formula VI is (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid.

In some embodiments of the processes herein, the compound of Formula VII is tert-butyl 5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate. In still other embodiments, the compound of Formula VII is tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (Compound 7*). In some embodiments, the compound of Formula VII is tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (Compound 7). In other embodiments, the compound of Formula VII is tert-butyl (1R,4R,5S)-5-(((Sₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate.

In some embodiments of the processes herein, the compound of Formula VIII is ethyl 4-chloro-6-(2-cyanoethyl)-7-(2,3-dichlorophenyl)-8-fluoro-2-methylquinoline-3-carboxylate (Compound 8a*). In still other embodiments, the compound of Formula VIII is ethyl (Rₐ)-4-chloro-6-(2-cyanoethyl)-7-(2,3-dichlorophenyl)-8-fluoro-2-methylquinoline-3-carboxylate (Compound 8a). In some embodiments, the compound of Formula VIII is ethyl (Sₐ)-4-chloro-6-(2-cyanoethyl)-7-(2,3-dichlorophenyl)-8-fluoro-2-methylquinoline-3-carboxylate.

In some embodiments of the processes herein, the compound of Formula VIII is ethyl 6-(2-cyanoethyl)-7-(2,3-dichlorophenyl)-8-fluoro-4-hydroxy-2-methylquinoline-3-carboxylate (Compound 8b*). In still other embodiments, the compound of Formula VIII is ethyl (Rₐ)-6-(2-cyanoethyl)-7-(2,3-dichlorophenyl)-8-fluoro-4-hydroxy-2-methylquinoline-3-carboxylate (Compound 8b). In some embodiments, the compound of Formula VIII is ethyl (Sₐ)-6-(2-cyanoethyl)-7-(2,3-dichlorophenyl)-8-fluoro-4-hydroxy-2-methylquinoline-3-carboxylate.

In some embodiments of the processes herein, the compound of Formula IX is tert-butyl 5-amino-2-azabicyclo[2.1.1]hexane-2-carboxylate. In some embodiments of the processes herein, the compound of Formula IX is tert-butyl (1R,4R,5S)-5-amino-2-azabicyclo[2.1.1]hexane-2-carboxylate (Compound 9).

In some embodiments of the processes herein, the compound of Formula X is ethyl 4-chloro-6-(2-cyanovinyl)-7-(2,3-dichlorophenyl)-8-fluoro-2-methylquinoline-3-carboxylate (Compound 10*). In some embodiments of the processes herein, the compound of Formula X is ethyl (Rₐ)-4-chloro-6-(2-cyanoethyl)-7-(2,3-dichlorophenyl)-8-fluoro-2-methylquinoline-3-carboxylate (Compound 10). In some embodiments of the processes herein, the compound of Formula X is ethyl (Sₐ)-4-chloro-6-(2-cyanoethyl)-7-(2,3-dichlorophenyl)-8-fluoro-2-methylquinoline-3-carboxylate.

In some embodiments of the processes herein, the compound of Formula X-A is ethyl 4-chloro-6-(2-cyanovinyl)-7-(2,3-dichlorophenyl)-8-fluoro-2-methylquinoline-3-carboxylate. In some embodiments of the processes herein, the compound of Formula X-A is ethyl (Rₐ)-4-chloro-6-(2-cyanoethyl)-7-(2,3-dichlorophenyl)-8-fluoro-2-methylquinoline-3-carboxylate. In some embodiments of the processes herein, the compound of Formula X-A is ethyl (Sₐ)-4-chloro-6-(2-cyanoethyl)-7-(2,3-dichlorophenyl)-8-fluoro-2-methylquinoline-3-carboxylate.

In some embodiments of the processes herein, the compound of Formula XI is ethyl 6-(2-cyanovinyl)-7-(2,3-dichlorophenyl)-8-fluoro-4-hydroxy-2-methylquinoline-3-carboxylate. In some embodiments of the processes herein, the compound of Formula XI is ethyl (Rₐ)-6-(2-cyanovinyl)-7-(2,3-dichlorophenyl)-8-fluoro-4-hydroxy-2-methylquinoline-3-carboxylate. In some embodiments of the processes herein, the compound of Formula XI is ethyl (Sₐ)-6-(2-cyanovinyl)-7-(2,3-dichlorophenyl)-8-fluoro-4-hydroxy-2-methylquinoline-3-carboxylate.

In some embodiments of the processes herein, the compound of Formula XII is ethyl 6-bromo-7-(2,3-dichlorophenyl)-8-fluoro-4-hydroxy-2-methylquinoline-3-carboxylate. In some embodiments of the processes herein, the compound of Formula XII is ethyl (Rₐ)-6-bromo-7-(2,3-dichlorophenyl)-8-fluoro-4-hydroxy-2-methylquinoline-3-carboxylate. In some embodiments of the processes herein, the compound of Formula XII is ethyl (Sₐ)-6-bromo-7-(2,3-dichlorophenyl)-8-fluoro-4-hydroxy-2-methylquinoline-3-carboxylate.

In some embodiments of the processes herein, the compound of Formula XIII is 6-bromo-7-(2,3-dichlorophenyl)-8-fluoro-2H-benzo[d][1,3]oxazine-2,4(1H)-dione. In some embodiments of the processes herein, the compound of Formula XIII is (Rₐ)-6-bromo-7-(2,3-dichlorophenyl)-8-fluoro-2H-benzo[d][1,3]oxazine-2,4(1H)-dione. In some embodiments of the processes herein, the compound of Formula XIII is (Sₐ)-6-bromo-7-(2,3-dichlorophenyl)-8-fluoro-2H-benzo[d][1,3]oxazine-2,4(1H)-dione.

In some embodiments of the processes herein, the compound of Formula XIV is ethyl 3-oxobutanoate.

In some embodiments of the processes herein, the compound of Formula XV is 3-amino-6-bromo-2′,3′-dichloro-2-fluoro-[1,1′-biphenyl]-4-carboxylic acid. In some embodiments of the processes herein, the compound of Formula XV is (Rₐ)-3-amino-6-bromo-2′,3′-dichloro-2-fluoro-[1,1′-biphenyl]-4-carboxylic acid. In some embodiments of the processes herein, the compound of Formula XV is (Sₐ)-3-amino-6-bromo-2′,3′-dichloro-2-fluoro-[1,1′-biphenyl]-4-carboxylic acid.

In some embodiments of the processes herein, the compound of Formula XVI is methyl 3-amino-6-bromo-2′,3′-dichloro-2-fluoro-[1,1′-biphenyl]-4-carboxylate. In some embodiments of the processes herein, the compound of Formula XVI is methyl (Rₐ)-3-amino-6-bromo-2′,3′-dichloro-2-fluoro-[1,1′-biphenyl]-4-carboxylate. In some embodiments of the processes herein, the compound of Formula XVI is methyl (Sₐ)-3-amino-6-bromo-2′,3′-dichloro-2-fluoro-[1,1′-biphenyl]-4-carboxylate.

In some embodiments of the processes herein, the compound of Formula XVII is methyl 3-amino-2′,3′-dichloro-2-fluoro-[1,1′-biphenyl]-4-carboxylate.

In some embodiments of the processes herein, the compound of Formula XVIII is methyl 2-amino-4-bromo-3-fluorobenzoate.

In some embodiments there is provided a process of using a compound of Formula I, II, III, IV, V, VI, VII, VIII, IX, X, XI, XII, XIII, XIV, XV, XVI, or XVII, or any of the embodiments thereof, in the manufacture of Compound 1*, Compound 1, Compound 1-HCl, or Compound-1-HCl·2H₂O. The process can include employing the compound as a feedstock for the manufacture. The process can include converting the compound to manufacture Compound 1*, Compound 1, Compound 1-HCl, or Compound-1-HCl·2H₂O.

In some embodiments there is provided a process of using Compound 1 in the manufacture of Compound 1-HCl, or Compound-1-HCl·2H₂O. The process can include employing Compound 1 as a feedstock for the manufacture. The process can include converting Compound 1 to manufacture Compound 1-HCl or Compound-1-HCl·2H₂O.

In some embodiments there is provided a process of Compound 2) in the manufacture of Compound 1, Compound 1-HCl, or Compound-1-HCl·2H₂O. The process can include employing Compound 1 as a feedstock for the manufacture. The process can include converting Compound 1 to manufacture Compound 1, Compound 1-HCl, or Compound-1-HCl·2H₂O.

In some embodiments there is provided a process of using Compound 3 in the manufacture of Compound 1, Compound 1-HCl, Compound-1-HCl·2H₂O, or Compound 2. The process can include employing Compound 3 as a feedstock for the manufacture. The process can include converting Compound 3 to manufacture Compound 1, Compound 1-HCl, Compound-1-HCl·2H₂O, or Compound 2.

In some embodiments there is provided a process of using Compound 4 in the manufacture of Compound 1, Compound 1-HCl, Compound-1-HCl·2H₂O, Compound 2, or Compound 3. The process can include employing Compound 4 as a feedstock for the manufacture. The process can include converting Compound 4 to manufacture Compound 1, Compound 1-HCl, Compound-1-HCl·2H₂O, Compound 2, or Compound 3.

In some embodiments there is provided a process of using Compound 5 in the manufacture of Compound 1, Compound 1-HCl, Compound-1-HCl·2H₂O, Compound 2, or Compound 3. The process can include employing Compound 5 as a feedstock for the manufacture. The process can include converting Compound 5 to manufacture Compound 1, Compound 1-HCl, Compound-1-HCl·2H₂O, Compound 2 or Compound 3.

In some embodiments there is provided a process of using Compound 6 in the manufacture of Compound 1, Compound 1-HCl, Compound-1-HCl·2H₂O, Compound 2, Compound 3 or Compound 5. The process can include employing Compound 6 as a feedstock for the manufacture. The process can include converting Compound 6 to manufacture Compound 1, Compound 1-HCl, Compound-1-HCl·2H₂O, Compound 2, Compound 3 or Compound 5.

In some embodiments there is provided a process of using Compound 7 in the manufacture of Compound 1, Compound 1-HCl, Compound-1-HCl·2H₂O, Compound 2, Compound 3, Compound 5 or Compound 6. The process can include employing Compound 7 as a feedstock for the manufacture. The process can include converting Compound 7 to manufacture Compound 1, Compound 1-HCl, Compound-1-HCl·2H₂O, Compound 2, Compound 3, Compound 5 or Compound 6.

In some embodiments there is provided a process of using Compound 8a in the manufacture of Compound 1, Compound 1-HCl, Compound-1-HCl·2H₂O, Compound 2, Compound 3, Compound 5, Compound 6 or Compound 7. The process can include employing Compound 8a as a feedstock for the manufacture. The process can include converting Compound 8a to manufacture Compound 1, Compound 1-HCl, Compound-1-HCl·2H₂O, Compound 2, Compound 3, Compound 5, Compound 6 or Compound 7.

In some embodiments there is provided a process of using Compound 8b in the manufacture of Compound 1, Compound 1-HCl, Compound-1-HCl·2H₂O, Compound 2, Compound 3, Compound 5, Compound 6 or Compound 7. The process can include employing Compound 8b as a feedstock for the manufacture. The process can include converting Compound 8b to manufacture Compound 1, Compound 1-HCl, Compound-1-HCl·2H₂O, Compound 2, Compound 3, Compound 5, Compound 6 or Compound 7.

In some embodiments there is provided a process of using Compound 9 in the manufacture of Compound 1, Compound 1-HCl, Compound-1-HCl·2H₂O, Compound 2, Compound 3, Compound 5, Compound 6 or Compound 7. The process can include employing Compound 9 as a feedstock for the manufacture. The process can include converting Compound 9 to manufacture Compound 1, Compound 1-HCl, Compound-1-HCl·2H₂O, Compound 2, Compound 3, Compound 5, Compound 6 or Compound 7.

In some embodiments there is provided a process of using Compound 10 in the manufacture of Compound 1, Compound 1-HCl, Compound-1-HCl·2H₂O, Compound 2, Compound 3, Compound 5, Compound 6, Compound 7, Compound 8a or Compound 8b. The process can include employing Compound 10 as a feedstock for the manufacture. The process can include converting Compound 10 to manufacture Compound 1, Compound 1-HCl, Compound-1-HCl·2H₂O, Compound 2, Compound 3, Compound 5, Compound 6, Compound 7, Compound 8a or Compound 8b.

In some embodiments, an atropisomer of the compound of Formula II, III, IV, V, VI, VII, VIII, IX or X is racemized to form a mixture of atropisomers of the corresponding compound. In other embodiments, the mixture of atropisomers of the compound of Formula II, III, IV, V, VI, VII, VIII, IX or X is separated into isolated stereoisomers of the corresponding compound. In other embodiments, one stereoisomer of the compound of Formula II, III, IV, V, VI, VII, VIII, IX or X is racemized to form a second mixture of stereoisomers of the corresponding compound. In other embodiments, the second mixture of stereoisomers of the compound of Formula II, III, IV, V, VI, VII, VIII, IX or X is separated into isolated stereoisomers of the corresponding compound. By performing iterative racemization and separation (i.e., one or more racemization and separation steps following an initial separation of a racemic mixture of atropisomers) an increased yield of one or other of the atropisomers (i.e., the Rₐ or Sₐ atropsomer) of the compound of Formula II, III, IV, V, VI, VII, VIII, IX or X can be obtained compared to a single chiral separation to isolate a single atropisomer from a racemic mixture.

The present disclosure also provides the full synthesis of a compound wherein multiple steps provided herein are combined in sequence. For example, provided herein is a process comprising forming a compound of Formula I from the starting material of a compound of Formula XX using the disclosed synthesis procedures. In some embodiments, the racemization can be carried out at a temperature in the range from about 0° C. to about 120° C., such as a temperature of about 5° C., about 10° C., about 15° C., about 20° C., about 25° C., about 30° C., about 40° C., about 50° C., about 60° C., about 70° C., about 80° C., about 90° C., about 100° C., about 110° C., or about 120° C. In some embodiments, the racemization can be carried out at a temperature from about 50° C. to about 90° C., or from about 60° C. to about 80° C., such as at about 70° C. In some embodiments, the racemization can be carried out at reflux.

In other embodiments, the racemization can be carried out in the presence of a suitable solvent, or mixtures thereof based on the solubility of the compounds described herein. Suitable solvents include, but are not limited to, DCM, THF, 1,4-dioxane, MeCN, toluene, tert-butanol, pyridine, N,N-diethylaniline, xylene, and DMSO, or combinations thereof.

In other embodiments, the process comprises chiral separation. In yet other embodiments, the process comprises separating atropisomers of a compound of Formula I, II, III, IV, V, VI, VII, VIII, IX, or X. In some embodiments, the separating can be performed using chromatography such as by HPLC or supercritical fluid chromatography. In some embodiments, the chromatography is performed using a chiral stationary phase. In some embodiments, the chiral stationary phase is a Pirkle type (Brush type) chiral stationary phase. In some embodiments, the chiral stationary phase is a protein-based chiral stationary phase. In some embodiments, the chiral stationary phase is a cyclodextrin-based chiral stationary phase. In some embodiments, the chiral stationary phase is a polymer-based carbohydrate chiral stationary phase (polysaccharide-based). In some embodiments, the chiral stationary phase is a macrocyclic antibiotic chiral stationary phase. In some embodiments, the chiral stationary phase is a chiral crown ether chiral stationary phase. In some embodiments, the chiral stationary phase is an imprinted polymer chiral stationary phase.

### III. Intermediate Compounds

In an aspect, provided herein is a compound of Formula II:

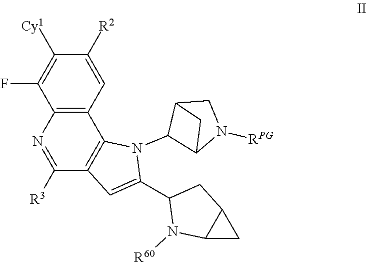

wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl.

In some embodiments, R² is CH₂CH₂CN.

In other embodiments, Cy¹ is 2,3-dichlorophenyl.

In yet other embodiments, R³ is methyl.

In still other embodiments, R⁶⁰ is C(O)R^{b60}. In some embodiments, R^{b60} is cyclopropyl.

In other embodiments, R^{PG} is tert-butyloxycarbonyl.

In yet other embodiments, the compound of Formula II is tert-butyl 5-(8-(2-cyanoethyl)-2-(2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate. In still other embodiments, the compound of Formula II is tert-butyl (1R,4R,5S)-5-(8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate. In some embodiments, wherein the compound of Formula II is tert-butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate. In other embodiments, the compound of Formula II is tert-butyl (1R,4R,5S)-5-((Sₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

In another aspect, provided herein is a compound of Formula III:

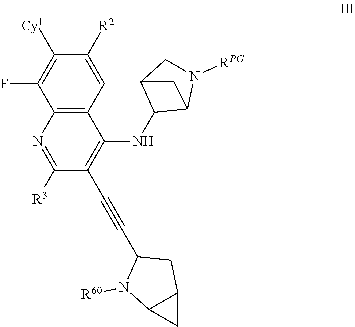

wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl.

In some embodiments, R² is CH₂CH₂CN.

In other embodiments, Cy¹ is 2,3-dichlorophenyl.

In yet other embodiments, R³ is methyl.

In still other embodiments, R⁶⁰ is C(O)R^{b60}. In some embodiments, R^{b60} is cyclopropyl.

In other embodiments, R^{PG} is tert-butyloxycarbonyl.

In yet other embodiments, the compound of Formula III is tert-butyl 5-((6-(2-cyanoethyl)-3-((2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate. In still other embodiments, the compound of Formula III is tert-butyl (1R,4R,5S)-5-((6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate. In some embodiments, the compound of Formula III is tert-butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate. In other embodiments, the compound of Formula III is tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate.

In yet another aspect, provided herein is a compound of Formula IV:

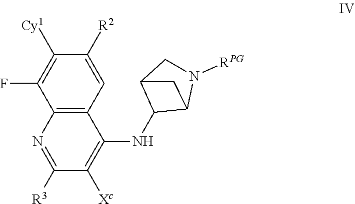

wherein
X^{c} is Cl, Br, or I;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, R² is CH₂CH₂CN.

In other embodiments, Cy¹ is 2,3-dichlorophenyl.

In yet other embodiments, R³ is methyl.

In still other embodiments, R^{PG} is tert-butyloxycarbonyl.

In some embodiments, the compound of Formula IV is tert-butyl 5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate. In other embodiments, the compound of Formula IV is tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate. In yet other embodiments, the compound of Formula IV is tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate. In still other embodiments, the compound of Formula IV is tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

In still other embodiments, provided herein is a compound of Formula VI:

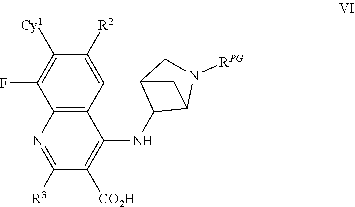

wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, R² is CH₂CH₂CN.

In other embodiments, Cy¹ is 2,3-dichlorophenyl.

In yet other embodiments, R³ is methyl.

In still other embodiments, R^{PG} is tert-butyloxycarbonyl.

In some embodiments, the compound of Formula VI is 4-((2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid. In other embodiments, the compound of Formula VI is 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid. In yet other embodiments, the compound of Formula VI is (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid. In still other embodiments, the compound of Formula VI is (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid.

In an aspect, provided herein is a compound of Formula VII:

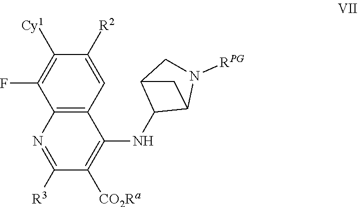

wherein
R^{a} is C₁₋₃ alkyl;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, R^{a} is ethyl.

In other embodiments, R² is CH₂CH₂CN.

In yet other embodiments, Cy¹ is 2,3-dichlorophenyl.

In still other embodiments, R³ is methyl.

In some embodiments, R^{PG} is tert-butyloxycarbonyl.

In other embodiments, the compound of Formula VII is tert-butyl 5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate. In yet other embodiments, the compound of Formula VII is tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate. In still other embodiments, the compound of Formula VII is tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate. In some embodiments, the compound of Formula VII is tert-butyl (1R,4R,5S)-5-(((Sₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate.

In another aspect, provided herein is a compound of Formula VIII:

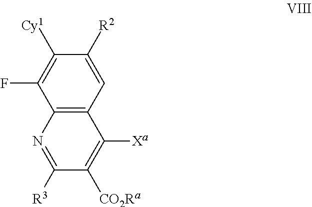

wherein
X^{a} is halo or OH;
R^{a} is C₁₋₃ alkyl;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, X^{a} is chloro.

In other embodiments, R^{a} is ethyl.

In yet other embodiments, R² is CH₂CH₂CN.

In still other embodiments, Cy¹ is 2,3-dichlorophenyl.

In some embodiments, R³ is methyl.

In other embodiments, the compound of Formula VIII is ethyl 4-chloro-6-(2-cyanoethyl)-7-(2,3-dichlorophenyl)-8-fluoro-2-methylquinoline-3-carboxylate. In yet other embodiments, the compound of Formula VIII is ethyl (Rₐ)-4-chloro-6-(2-cyanoethyl)-7-(2,3-dichlorophenyl)-8-fluoro-2-methylquinoline-3-carboxylate. In still other embodiments, the compound of Formula VIII is ethyl (Sₐ)-4-chloro-6-(2-cyanoethyl)-7-(2,3-dichlorophenyl)-8-fluoro-2-methylquinoline-3-carboxylate.

In yet another aspect, provided herein is a compound of Formula X:

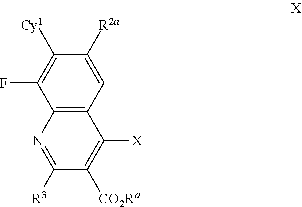

wherein
R^{a} is C₁₋₃ alkyl;
X is halo;
R^{2a} is selected from C₂₋₄ alkyl and C₂₋₄ alkenyl, both of which are optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, X^{a} is chloro.

In other embodiments, R^{a} is ethyl.

In yet other embodiments, R^{2a} is CH═CHCN.

In still other embodiments, Cy¹ is 2,3-dichlorophenyl.

In some embodiments, R³ is methyl.

In other embodiments, the compound of Formula X is ethyl 4-chloro-6-(2-cyanovinyl)-7-(2,3-dichlorophenyl)-8-fluoro-2-methylquinoline-3-carboxylate. In some embodiments of the processes herein, the compound of Formula X is ethyl (Rₐ)-4-chloro-6-(2-cyanoethyl)-7-(2,3-dichlorophenyl)-8-fluoro-2-methylquinoline-3-carboxylate. In some embodiments of the processes herein, the compound of Formula X is ethyl (Sₐ)-4-chloro-6-(2-cyanoethyl)-7-(2,3-dichlorophenyl)-8-fluoro-2-methylquinoline-3-carboxylate.

In still another aspect, provided herein is a compound of Formula XI:

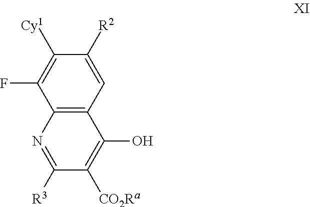

wherein
R^{a} is C₁₋₃ alkyl;
Cy¹ is phenyl; wherein the phenyl is optionally substituted with 1 or 2 substituents independently selected from R¹⁰;
R² is selected from C₂₋₄ alkenyl optionally substituted with CN;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, R^{a} is ethyl.

In other embodiments, R^{2a} is CH═CHCN.

In yet other embodiments, Cy¹ is 2,3-dichlorophenyl.

In still other embodiments, R³ is methyl.

In some embodiments, the compound of Formula XI is ethyl 6-(2-cyanovinyl)-7-(2,3-dichlorophenyl)-8-fluoro-4-hydroxy-2-methylquinoline-3-carboxylate. In some embodiments of the processes herein, the compound of Formula XI is ethyl (Rₐ)-6-(2-cyanovinyl)-7-(2,3-dichlorophenyl)-8-fluoro-4-hydroxy-2-methylquinoline-3-carboxylate. In some embodiments of the processes herein, the compound of Formula XI is ethyl (Sₐ)-6-(2-cyanovinyl)-7-(2,3-dichlorophenyl)-8-fluoro-4-hydroxy-2-methylquinoline-3-carboxylate.

In an aspect, provided herein is a compound of Formula XII:

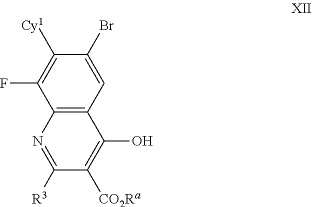

wherein
R^{a} is C₁₋₃ alkyl;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D.

In some embodiments, R^{a} is ethyl.

In other embodiments, Cy¹ is 2,3-dichlorophenyl.

In yet other embodiments, R³ is methyl.

In still other embodiments, the compound of Formula XII is ethyl 6-bromo-7-(2,3-dichlorophenyl)-8-fluoro-4-hydroxy-2-methylquinoline-3-carboxylate. In some embodiments of the processes herein, the compound of Formula XII is ethyl (Rₐ)-6-bromo-7-(2,3-dichlorophenyl)-8-fluoro-4-hydroxy-2-methylquinoline-3-carboxylate. In some embodiments of the processes herein, the compound of Formula XII is ethyl (Sₐ)-6-bromo-7-(2,3-dichlorophenyl)-8-fluoro-4-hydroxy-2-methylquinoline-3-carboxylate.

In another aspect, provided herein is a compound of Formula XIII:

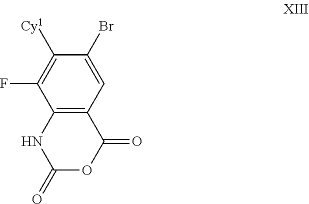

wherein
Cy¹ is phenyl; wherein the phenyl is optionally substituted with 1 or 2 substituents independently selected from R¹⁰; and
each R¹⁰ is independently selected from C₁₋₃ alkyl and halo.

In some embodiments, Cy¹ is 2,3-dichlorophenyl.

In other embodiments, the compound of Formula XIII is 6-bromo-7-(2,3-dichlorophenyl)-8-fluoro-2H-benzo[d][1,3]oxazine-2,4(1H)-dione. In some embodiments of the processes herein, the compound of Formula XIII is (Rₐ)-6-bromo-7-(2,3-dichlorophenyl)-8-fluoro-2H-benzo[d][1,3]oxazine-2,4(1H)-dione. In some embodiments of the processes herein, the compound of Formula XIII is (Sₐ)-6-bromo-7-(2,3-dichlorophenyl)-8-fluoro-2H-benzo[d][1,3]oxazine-2,4(1H)-dione.

In yet another aspect, provided herein is a compound of Formula XV:

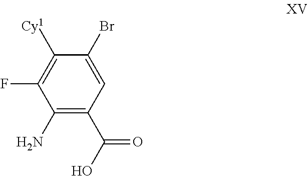

wherein
Cy¹ is phenyl; wherein the phenyl is optionally substituted with 1 or 2 substituents independently selected from R¹⁰; and
each R¹⁰ is independently selected from C₁₋₃ alkyl and halo.

In some embodiments, Cy¹ is 2,3-dichlorophenyl.

In other embodiments, the compound of Formula XV is 3-amino-6-bromo-2′,3′-dichloro-2-fluoro-[1,1′-biphenyl]-4-carboxylic acid. In some embodiments of the processes herein, the compound of Formula XV is (Rₐ)-3-amino-6-bromo-2′,3′-dichloro-2-fluoro-[1,1′-biphenyl]-4-carboxylic acid. In some embodiments of the processes herein, the compound of Formula XV is (Sₐ)-3-amino-6-bromo-2′,3′-dichloro-2-fluoro-[1,1′-biphenyl]-4-carboxylic acid.

In still another aspect, provided herein is a compound of Formula XVI:

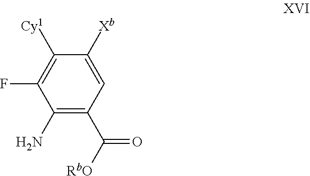

wherein
X^{b} is Cl, Br, or I;
R^{b} is C₁₋₃ alkyl;
Cy¹ is phenyl; wherein the phenyl is optionally substituted with 1 or 2 substituents independently selected from R¹⁰; and
each R¹⁰ is independently selected from C₁₋₃ alkyl and halo.

In some embodiments, R^{b} is methyl.

In other embodiments, Cy¹ is 2,3-dichlorophenyl.

In yet other embodiments, the compound of Formula XVI is methyl 3-amino-6-bromo-2′,3′-dichloro-2-fluoro-[1,1′-biphenyl]-4-carboxylate. In some embodiments of the processes herein, the compound of Formula XVI is methyl (Rₐ)-3-amino-6-bromo-2′,3′-dichloro-2-fluoro-[1,1′-biphenyl]-4-carboxylate. In some embodiments of the processes herein, the compound of Formula XVI is methyl (Sₐ)-3-amino-6-bromo-2′,3′-dichloro-2-fluoro-[1,1′-biphenyl]-4-carboxylate.

In an aspect, provided herein is a compound of Formula XVII:

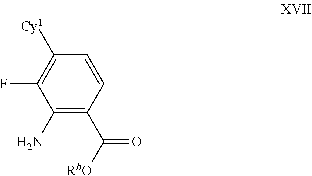

wherein
Cy¹ is phenyl; wherein the phenyl is optionally substituted with 1 or 2 substituents independently selected from R¹⁰;
each R¹⁰ is independently selected from C₁₋₃ alkyl and halo; and
R^{b} is C₁₋₃ alkyl.

In some embodiments, R^{b} is methyl.

In other embodiments, Cy¹ is 2,3-dichlorophenyl.

In yet other embodiments, the compound of Formula XVII is methyl 3-amino-2′,3′-dichloro-2-fluoro-[1,1′-biphenyl]-4-carboxylate.

In some embodiments of the formulae herein, each R¹⁰ is independently selected from halo. In other embodiments of the formulae herein, each R¹⁰ is chloro.

Those skilled in the art will recognize or be able to ascertain using no more than routine experimentation, numerous equivalents to the specific procedures, embodiments, claims, and examples described herein. Such equivalents are within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The disclosure is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1: Synthesis of 3-((Rₐ)-1-((1R,4R,5S)-2-Azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1)

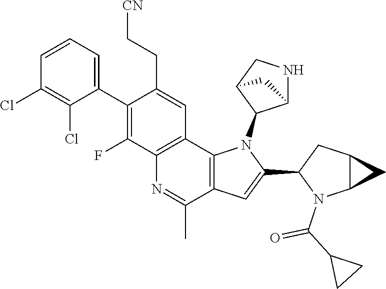 [SMILES: Cc1nc2c(F)c(-c3cccc(Cl)c3Cl)c(CCC#N)cc2c2c1cc([C@H]1C[C@H]3C[C@H]3N1C(=O)C1CC1)n2[C@H]1[C@H]2CN[C@@H]1C2]

### Step 1. Methyl 2-amino-4-bromo-3-fluorobenzoate

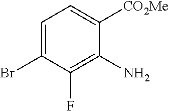 [SMILES: COC(=O)c1ccc(Br)c(F)c1N]

Dimethyl sulfate (823 g, 6.53 mol) was added to a mixture of 2-amino-4-bromo-3-fluorobenzoic acid (1500 g, 6.22 mol) and K₂CO₃ (945 g, 6.84 mol) in DMF or 1,4-dioxane (6 L) at 5-50° C. After the addition, the mixture was stirred at r.t. for 2 h to complete the reaction. Water (7.5 L) was gradually added to the reaction mixture to precipitate the product. After the water addition, the mixture was stirred at r.t. for 1 h. The solids were isolated by filtration and the wet cake was washed with water (3×1.5 L). The solids were dried under vacuum at about 50° C. overnight to give the title compound (1530 g, 99% yield). LCMS calc. for C₈H₇BrFNO₂: 246.96; Found: 248 (M+H⁺). ¹H NMR (400 MHz, DMSO-d₆) δ 7.49 (dd, J=8.8, 1.7 Hz, 1H), 6.87-6.77 (m, 3H), 3.82 (s, 3H). ¹⁹F NMR (376 MHz, DMSO-d₆) δ −127.24

### Step 2. Methyl 3-amino-2′,3′-dichloro-2-fluoro-[1,1′-biphenyl]-4-carboxylate

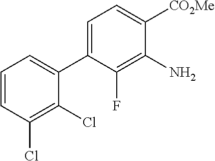 [SMILES: COC(=O)c1ccc(-c2cccc(Cl)c2Cl)c(F)c1N]

Bis(di-tert-butyl(4-dimethylaminophenyl)phosphine)dichloropalladium(II) (Pd-132) (8.12 g, 0.011 mol) was added to a mixture of methyl 2-amino-4-bromo-3-fluorobenzoate (1420 g, 5.72 mol), 2,3-dichlorophenylboronic acid (1226 g, 6.3 mol) and potassium fluoride (732 g, 12.6 mol) in MeCN (6 L) and water (1.5 L). The mixture was degassed and refilled with nitrogen and heated to 70° C. for 1 h to complete the reaction. Water (6 L) was added to the reaction mixture at 50° C. The mixture was cooled to r.t. and stirred for 1 h. The solids were isolated by filtration and the wet cake was washed with 50% MeCN in water (2×2 L) and water (2×2 L). The solids were dried under vacuum at about 50° C. overnight to give the title compound (1700 g, 94% yield). LCMS calc. for C₁₄H₉Cl₂FNO₂: 313.01; Found: 314 (M+H⁺). ¹H NMR (400 MHz, DMSO-d₆) δ 7.74 (dd, J=8.0, 1.6 Hz, 1H), 7.64 (dd, J=8.4, 1.4 Hz, 1H), 7.48 (t, J=7.9 Hz, 1H), 7.40 (dd, J=7.9, 1.6 Hz, 1H), 6.70 (s(b), 2H), 6.51 (dd, J=8.3, 6.6 Hz, 1H), 3.86 (s, 3H). ¹⁹F NMR (376 MHz, DMSO-d₆) δ −134.70

### Step 3. Methyl 3-amino-6-bromo-2′,3′-dichloro-2-fluoro-[1,1′-biphenyl]-4-carboxylate

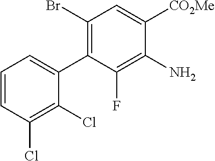 [SMILES: COC(=O)c1cc(Br)c(-c2cccc(Cl)c2Cl)c(F)c1N]

N-Bromosuccinimide (684 g, 3.84 mol) was added to a solution of methyl 3-amino-2′,3′-dichloro-2-fluoro-[1,1′-biphenyl]-4-carboxylate (1150 g, 3.66 mol) in MeCN (5.75 L) at 50-66° C. After the reaction completion, the MeCN (3 L) was removed by rotary evaporation. Water (5.75 L) was added to the concentrated mixture and the resulting mixture was stirred at r.t. for 2-3 h. The solids were isolated by filtration and the wet cake was washed with water to give methyl 3-amino-6-bromo-2′,3′-dichloro-2-fluoro-[1,1′-biphenyl]-4-carboxylate. LCMS calc. for C₁₄H₉BrFCl₂NO₂: 390.92; Found: 391 (M+H). ¹H NMR (400 MHz, DMSO-d₆) δ 7.86 (d, J=1.7 Hz, 1H), 7.79 (dd, J=8.1, 1.5 Hz, 1H), 7.52 (t, J=7.9 Hz, 1H), 7.40 (dd, J=7.7, 1.5 Hz, 1H), 6.83 (s(b), 2H), 3.87 (s, 3H). ¹⁹F NMR (376 MHz, DMSO-d₆) δ −128.19.

### Step 4. 3-Amino-6-bromo-2′,3′-dichloro-2-fluoro-[1,1′-biphenyl]-4-carboxylic acid

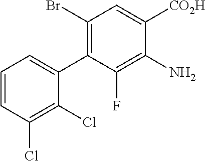 [SMILES: Nc1c(C(=O)O)cc(Br)c(-c2cccc(Cl)c2Cl)c1F]

The wet cake of methyl 3-amino-6-bromo-2′,3′-dichloro-2-fluoro-[1,1′-biphenyl]-4-carboxylate was dissolved in THE (3 L) and MeOH (1.5 L). Aq. NaOH (1.5 M; 5 L) was added to the solution and the mixture was stirred at about 50° C. for 2 h to complete the saponification reaction. Aq. HCl (1.5 M) was gradually added to the mixture to adjust the pH to 3-4 and the mixture was stirred at r.t. for 1 h. The solids were isolated by filtration and the wet cake was washed with water (3×1.2 L). The solids were dried under vacuum at about 50° C. overnight to give the title compound (1354 g, 97.5% yield over two steps). LCMS calc. for C₁₃H₇BrCl₂FNO₂: 376.90; Found: 378 (M+H⁺). ¹H NMR (400 MHz, DMSO-d₆) δ 7.85 (d, J=1.7 Hz, 1H), 7.78 (dd, J=8.1, 1.5 Hz, 1H), 7.52 (t, J=7.9 Hz, 1H), 7.39 (dd, J=7.9, 1.5 Hz, 1H), 6.88. ¹⁹F NMR (376 MHz, DMSO-d₆) δ −128.95.

### Step 5. 6-Bromo-7-(2,3-dichlorophenyl)-8-fluoro-2H-benzo[d][1,3]oxazine-2,4(1H)-dione

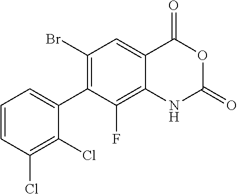 [SMILES: O=c1[nH]c2c(F)c(-c3cccc(Cl)c3Cl)c(Br)cc2c(=O)o1]

Triphosgene (500 g, 1.65 mol) in THE (500 mL) was added to the solution of 3-amino-6-bromo-2′,3′-dichloro-2-fluoro-[1,1′-biphenyl]-4-carboxylic acid (1254 g, 3.31 mol) in THE (4 L) at 60° C. and the mixture was stirred for 1 h to complete the reaction. The mixture was cooled to 35° C. and n-heptane (10 L) was slowly charged to precipitate the product. The mixture was cooled to r.t. and stirred for 1 h. The solids were isolated by filtration and washed with n-heptane (2×1 L). The wet cake was dried under vacuum at about 50° C. overnight to give the title compound (1385 g, quantitative yield). LCMS calc. for C₁₄H₅BrCl₂FNO₃: 402.88; Found: 404 (M+H⁺). ¹H NMR (400 MHz, DMSO-d₆) δ 12.24 (s, 1H), 8.10 (d, J=1.5 Hz, 1H), 7.85 (dd, J=8.1, 1.5 Hz, 1H), 7.58 (t, J=7.9 Hz, 1H), 7.43 (dd, J=7.7, 1.5 Hz, 1H). ¹⁹F NMR (376 MHz, DMSO-d₆) δ −123.98.

### Step 6. Ethyl 6-bromo-7-(2,3-dichlorophenyl)-8-fluoro-4-hydroxy-2-methylquinoline-3-carboxylate

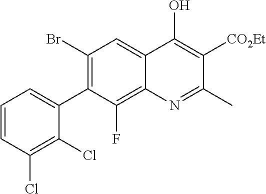 [SMILES: CCOC(=O)c1c(C)nc2c(F)c(-c3cccc(Cl)c3Cl)c(Br)cc2c1O]

A mixture of 6-bromo-7-(2,3-dichlorophenyl)-8-fluoro-2H-benzo[d][1,3]oxazine-2,4(1H)-dione (1078 g, 2.66 mol), ethyl acetoacetate (693 g, 5.32 mol), sodium acetate (393 g, 4.79 mol) and sodium chloride (933 g. 16 mol) in DMSO (5 L) was heated to 50-60° C. for 5 h. The temperature was raised to 100° C. and the mixture was stirred for 1 h to complete the reaction. The mixture was cooled to about 60° C. and water (10 L) was gradually added to precipitate the product. The mixture was cooled to r.t. and stirred for 1 h. The solids were isolated by filtration and the wet cake was washed with water (2×2 L). The wet solids were dried under vacuum at about 50° C. overnight to give the title compound (1145 g, 91% yield). LCMS calc. for C₁₉H₁₃BrCl₂FNO₂: 470.94; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 6b. Ethyl 6-bromo-7-(2,3-dichlorophenyl)-8-fluoro-4-hydroxy-2-methylquinoline-3-carboxylate

The title compound can alternatively be prepared by the following process. A solution of methyl 3-amino-2′,3′-dichloro-2-fluoro-[1,1′-biphenyl]-4-carboxylate (100 g, 0.254 mol), ethyl acetoacetate (33.1 g, 0.51 mol) and p-toluenesulfonic acid (2.2 g, 0.013 mol) in xylene (1 L) was refluxed for 5 h to azeotropically remove water. Sodium ethoxide (26 g, 0.381 mol) was added to the mixture and the mixture was refluxed for another 5 h. The mixture was cooled to r.t. and poured into dilute HCl pH=6-7. The organic phase was separated, and the aqueous phase was extracted with EtOAc. The combined organic phases were concentrated, and the product was purified over silica gel column and eluted with EtOAc and heptane (0-30%) to give the title compound (65 g, 54%). LCMS calc. for C₁₉H₁₃BrCl₂FNO₃: 470.91; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 7. Ethyl 6-(2-cyanovinyl)-7-(2,3-dichlorophenyl)-8-fluoro-4-hydroxy-2-methylquinoline-3-carboxylate

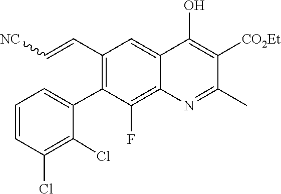 [SMILES: CCOC(=O)c1c(C)nc2c(F)c(-c3cccc(Cl)c3Cl)c(C=CC#N)cc2c1O]

A mixture of ethyl 6-bromo-7-(2,3-dichlorophenyl)-8-fluoro-4-hydroxy-2-methylquinoline-3-carboxylate (246 g, 0.52 mol), acrylonitrile (69 g, 1.3 mol), NEt₃ (156 g, 1.56 mol) and bis(di-tert-butyl)-(4-dimethylaminophenyl)phosphine)dichloridopalladium (II) (Pd-132) (14.7 g, 0.02 mol) in DMF (1.5 L) was heated to 85° C. for about 5 h to complete the reaction. The mixture was cooled to 50° C. and water (1 L) was gradually added. The mixture was cooled to r.t. and aq. HCl (1 M) was added to adjust the pH to pH 5-6. The solids were isolated by filtration and the wet cake was washed with water (2×500 mL). The wet solids were dissolved in MeOH (1 L) and DCM (9 L). To the solution was added sodium bisulfite (186 g, 1.8 mol) and water (4 L). The mixture was stirred at r.t. for 1 h and the aqueous phase was separated and discarded. The organic phase was washed with water (2×2 L). Activated charcoal (150 g) was added to the organic solution and the mixture was stirred at r.t. for 1 h. The mixture was filtered over a diatomaceous earth bed and the bed was rinsed with DCM (2 L). The organic solution was concentrated to about 1 L and heptane (3.5 L) was gradually added to precipitate the product. The solids were isolated by filtration and washed with heptane (2×2 L). The wet solids were dried under vacuum at about 50° C. overnight to give the title compound (210 g, 90% yield). LCMS calc. for C₂₂H₁₅Cl₂FNO₃: 444.04; Found: 445 (M+H⁺). ¹H-NMR (400 MHz, DMSO-d₆) (cis and trans mixture): δ 12.05 (s, 1H), 8.64 (s, OH), 8.39 (s, 1H), 7.86 (td, J=7.7, 1.5 Hz, 1H), 7.63-7.53 (m, 1H), 7.47 (td, J=7.5, 1.6 Hz, 1H), 7.04 (d, J=16.5 Hz, 1H), 6.88 (d, J=11.9 Hz, OH), 6.55 (d, J=16.6 Hz, 1H), 5.91 (d, J=12.0 Hz, OH), 4.29 (q, J=7.1 Hz, 2H), 2.47 (d, J=5.0 Hz, 4H), 1.30 (td, J=7.1, 3.2 Hz, 4H).

### Step 8. Ethyl 6-(2-cyanoethyl)-7-(2,3-dichlorophenyl)-8-fluoro-4-hydroxy-2-methylquinoline-3-carboxylate

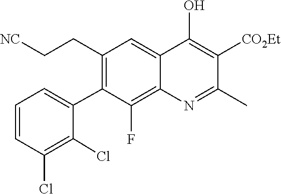 [SMILES: CCOC(=O)c1c(C)nc2c(F)c(-c3cccc(Cl)c3Cl)c(CCC#N)cc2c1O]

A mixture of ethyl 6-(2-cyanovinyl)-7-(2,3-dichlorophenyl)-8-fluoro-4-hydroxyl-2-methylquinoline-3-carboxylate (155 g, 348 mmol), pyridine (450 mL) and 1,4-dioxane (450 mL) was heated to 50-60° C. to give a homogenous solution. To the solution was added NaBH₄ (65.8 g, 1741 mmol) in portions at 50-60° C. The resulting mixture was stirred for 22 h at 50-60° C. to complete the reduction. After cooling to about 15° C., EtOAc (950 mL) was added to the reaction mixture. Concentrated HCl was gradually added to the mixture to adjust the aqueous phase pH to 1-2. The organic phase was separated, and the aqueous phase was extracted with EtOAc (500 mL). The combined EtOAc phase was washed with aq. HCl (1 M, 500 mL), water (2×500 mL), 10% brine (300 mL) and dried over sodium sulfate (75 g). The solution was concentrated, and the residue was purified by silica gel column (0-20% MeOH in DCM) to give the title compound (117.8 g, 76%). LCMS calc. for C₂₂H₁₇Cl₂FN₂O₃: 446.06; Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Step 9. Ethyl 4-chloro-6-(2-cyanoethyl)-7-(2,3-dichlorophenyl)-8-fluoro-2-methylquinoline-3-carboxylate

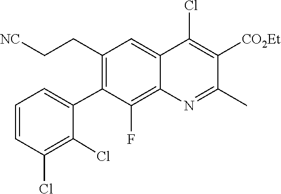 [SMILES: CCOC(=O)c1c(C)nc2c(F)c(-c3cccc(Cl)c3Cl)c(CCC#N)cc2c1Cl]

A mixture of ethyl 6-(2-cyanoethyl)-7-(2,3-dichlorophenyl)-8-fluoro-4-hydroxy-2-methylquinoline-3-carboxylate (60 g, 134 mmol), benzyltriethylammonium chloride (31 g, 135 mmol), N,N-dimethylaniline (49.1 g, 405 mmol) in MeCN (300 mL) was added phosphorus oxychloride (62 g, 405 mmol) at below 20° C. The mixture was heated to 60° C. for 1 h to complete the reaction. The mixture was cooled to r.t. and pooled into ice-water (900 mL) at a temperature below 20° C. Product precipitated out during the aqueous quench. The mixture was stirred at r.t. for more than 5 h. The solids were isolated by filtration and the wet cake was washed with 10% MeCN in water (2×150 mL). The wet solids were dried under vacuum at about 50° C. overnight to give the title compound (57 g, 90% yield). LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 10. Ethyl 4-chloro-6-(2-cyanovinyl)-7-(2,3-dichlorophenyl)-8-fluoro-2-methylquinoline-3-carboxylate

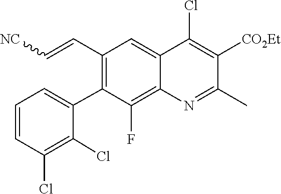 [SMILES: CCOC(=O)c1c(C)nc2c(F)c(-c3cccc(Cl)c3Cl)c(C=CC#N)cc2c1Cl]

A mixture of ethyl 6-(2-cyanovinyl)-7-(2,3-dichlorophenyl)-8-fluoro-4-hydroxy-2-methylquinoline-3-carboxylate (600 g, 1.35 mol), benzyltriethylammonium chloride (307 g, 1.35 mol), N,N-diethylaniline (603 g, 4.04 mol) in MeCN (3 L) was added phosphorus oxychloride (389.7 g, 4.04 mol) at below 20° C. The mixture was heated to 60° C. for 1 h to complete the reaction. The mixture was cooled to r.t. and pooled into ice-water (9 L) at a temperature below 20° C. Product precipitated out during the aqueous quench. The mixture was stirred at r.t. for more than 5 h. The solids were isolated by filtration and the wet cake was washed with 10% MeCN in water (2×1.5 L). The wet solids were dried under vacuum at about 50° C. overnight to give the title compound (563 g, 90% yield). LCMS calc. for C₂₂H₁₄Cl₃FN₂O₂: 462.01; Found: 463 (M+H⁺). ¹H-NMR (400 MHz, DMSO-d₆) (mixture of cis and trans isomers) δ 8.72 (s, 0.3H), 8.51 (s, 1H), 7.87 (ddd, J=7.3, 5.6, 1.5 Hz, 1.3H), 7.64-7.46 (m, 3H), 7.21 (d, J=16.5 Hz, 1H), 7.05 (d, J=11.9 Hz, 0.3H), 6.73 (d, J=16.5 Hz, 1H), 6.08 (d, J=11.9 Hz, 0.3H), 4.53 (qd, J=7.1, 2.0 Hz, 2H), 2.72 (d, J=7.4 Hz, 4H), 1.41 (t, J=7.1 Hz, 4H).

### Step 11. Ethyl 4-chloro-6-(2-cyanoethyl)-7-(2,3-dichlorophenyl)-8-fluoro-2-methylquinoline-3-carboxylate

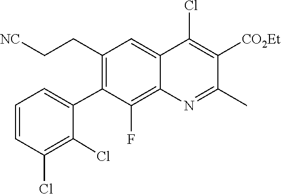 [SMILES: CCOC(=O)c1c(C)nc2c(F)c(-c3cccc(Cl)c3Cl)c(CCC#N)cc2c1Cl]

A mixture of ethyl 4-chloro-6-(2-cyanovinyl)-7-(2,3-dichlorophenyl)-8-fluoro-2-methylquinoline-3-carboxylate (528 g, 1.14 mol) and PMHS (411 g, 6.83 mol) in toluene (1.8 L) was stirred at about 50° C. In another 2-L flask, a mixture of diacetoxycopper hydrate (4.1 g, 0.02 mol), (9,9-dimethyl-9H-xanthene-4,5-diyl)bis(diphenylphosphane) (13.58 g, 0.023 mol) in toluene (300 mL) and tert-butanol (483 g, 6.52 mol) was stirred for 1-2 h to form a solution. The copper acetate solution was slowly added to the solution of ethyl 4-chloro-6-(2-cyanovinyl)-7-(2,3-dichlorophenyl)-8-fluoro-2-methylquinoline-3-carboxylate and PMHS in toluene at 50-60° C. to complete the reduction. The reaction mixture was concentrated under vacuum distillation to about 2 L. To the 2 L residue was added heptane (8 L) at about 50° C. for 1 h. The mixture was cooled to r.t. and stirred overnight. The solids were isolated by filtration and the wet cake was washed with heptane (2×1.2 L). The wet cake and silica gel (260 g) in DCM (2.7 L) were stirred for 1 h. The mixture was filtered over a silica gel bed (260 g) and the silica gel bed was rinsed with DCM (4 L) until the eluent was almost colorless. The DCM was removed. DCM (140 mL) and MTBE (260 mL) were added to the residue. The solids were isolated by filtration and the wet cake was washed with MTBE (2×1.2 L). The wet solids were dried under vacuum at about 50° C. overnight to give the title compound (476 g, 90% yield). LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 12. Ethyl (Rₐ)-4-chloro-6-(2-cyanoethyl)-7-(2,3-dichlorophenyl)-8-fluoro-2-methylquinoline-3-carboxylate and ethyl (Sₐ)-4-chloro-6-(2-cyanoethyl)-7-(2,3-dichlorophenyl)-8-fluoro-2-methylquinoline-3-carboxylate

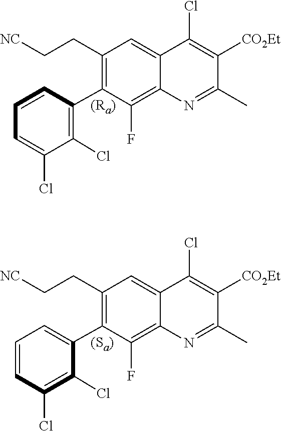

The racemic ethyl 4-chloro-6-(2-cyanoethyl)-7-(2,3-dichlorophenyl)-8-fluoro-2-methylquinoline-3-carboxylate was subject to chiral separation (CHIRALPAK® IB N, MTBE as eluent) to give both ethyl (Rₐ)-4-chloro-6-(2-cyanoethyl)-7-(2,3-dichlorophenyl)-8-fluoro-2-methylquinoline-3-carboxylate and ethyl (Sₐ)-4-chloro-6-(2-cyanoethyl)-7-(2,3-dichlorophenyl)-8-fluoro-2-methylquinoline-3-carboxylate. LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H)

### Step 13. Ethyl 4-chloro-6-(2-cyanoethyl)-7-(2,3-dichlorophenyl)-8-fluoro-2-methylquinoline-3-carboxylate by Racemization

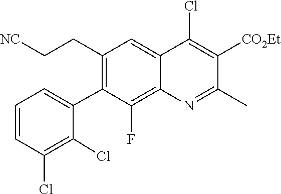 [SMILES: CCOC(=O)c1c(C)nc2c(F)c(-c3cccc(Cl)c3Cl)c(CCC#N)cc2c1Cl]

A mixture of ethyl (Sₐ)-4-chloro-6-(2-cyanoethyl)-7-(2,3-dichlorophenyl)-8-fluoro-2-methylquinoline-3-carboxylate (100 g) in sulfolane (200 mL) was heated to 185° C. for 2 h to give racemic ethyl 4-chloro-6-(2-cyanoethyl)-7-(2,3-dichlorophenyl)-8-fluoro-2-methylquinoline-3-carboxylate. The mixture was cooled to 50° C. and MeCN (200 mL) was added. To the solution was added water (700 mL) at 50° C. The mixture was cooled to r.t. and stirred for 4 h. The solids were isolated by filtration and the wet cake was washed with water (2×200 mL). The wet solids were dried under vacuum at about 50° C. overnight to give the title compound (97 g, 97% yield). LCMS calc. for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

The alternative atropisomer ethyl-(Sₐ)-4-chloro-6-(2-cyanoethyl)-7-(2,3-dichlorophenyl)-8-fluoro-2-methylquinoline-3-carboxylate is convertible to a racemic mixture using an analogous process.

### Step 14. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

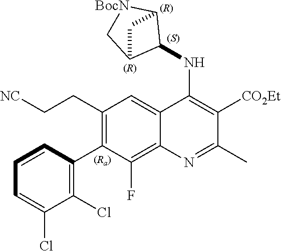

A mixture of ethyl (Rₐ)-4-chloro-6-(2-cyanoethyl)-7-(2,3-dichlorophenyl)-8-fluoro-2-methylquinoline-3-carboxylate (106.3 g, 228 mmol), tert-butyl (1R,4R,5S)-5-amino-2-azabicyclo[2.1.1]hexane-2-carboxylate (58.8 g, 297 mmol), lithium chloride (19 g, 446 mmol), and DIPEA (99.5 g, 670 mmol) in DMSO (400 mL) was heated to 80° C. overnight. The reaction mixture was cooled to r.t. and MTBE (1 L) and water (500 mL) were subsequently added. The organic phase was separated. The organic phase was washed with aq. HCl (0.1 M, 500 mL), sat. aq. NaHCO₃ (500 mL) and water (500 mL). The solvent was removed under reduced pressure to give the title compound that was used for next step without further purification. An analytical sample was purified by silica gel column (0-10% MeOH in DCM). LCMS calc. for C₃₂H₃₃Cl₂FN₄O₄: 626.19; Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J=4.1 Hz, 1H), 3.65-3.43 (m, 1H), 3.18 (d, J=9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 9H).

The alternative atropisomer tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 14a. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

The title compound can be alternatively prepared by the following method. A mixture of ethyl (R)-4-chloro-6-(2-cyanoethyl)-7-(2,3-dichlorophenyl)-8-fluoro-2-methylquinoline-3-carboxylate (40 g, 85 mmol), lithium carbonate (19 g, 258 mmol), and tert-Butyl (1R,4R,5S)-5-amino-2-azabicyclo[2.1.1]hexane-2-carboxylate oxalate (29.4 g, 98 mmol) in DMSO (120 mL) was heated to 80° C. overnight. The reaction mixture was cooled to r.t. and MTBE (300 mL) and filtered. The solids were rinsed with MTBE (100 mL). The combined filtrate was washed with water (2×320 mL). The organic phase was separated. The solvent was removed under reduced pressure to give the product that was used for next step without further purification. An analytical sample was purified by silica gel column (0-10% MeOH in DCM). LCMS calc. for C₃₂H₃₃Cl₂FN₄O₄: 626.19; Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J==4.1 Hz, 1H), 3.65-3.43 (m, 1H), 3.18 (d, J==9.3 Hz, 1H), 3.02 (s, 1H), 2.91-2.74 (m, 2H), 2.70 (dd, J==13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 6H).

The alternative atropisomer tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

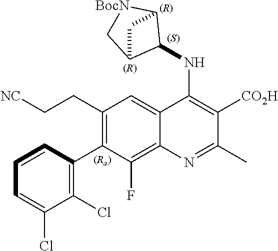

Aq. NaOH (2 M; 134 mL, 268 mmol) was added to a solution of tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (140.0 g, 223 mmol) in MeCN (560 mL) and MeOH (210 mL) at r.t. The mixture was heated to 50° C. for 1-1.5 h. The mixture was cooled to r.t. and acidified with aq. HCl (1 M) to about pH 5. The MeCN and MeOH were removed under vacuum. The product was extracted by EtOAc (1.7 L). The aqueous phase was separated and extracted with EtOAc (420 mL). The combined EtOAc phases were concentrated under vacuum to give a residue. MTBE (300 mL) was added to the residue and the mixture slurry was agitated at r.t. for 2 h. The solids were isolated by filtration and the wet cake was washed with MTBE (2×100 mL). The solids were dried under vacuum at about 50° C. to give the title compound (135 g, quantitative) that was used for next step without further purification.

The alternative atropisomer (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid is prepared by an analogous route by performing processes analogous to Steps 14 and 15b starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15b. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

The title compound can be alternatively prepared by the following process. Sodium trimethylsilanolate (338 g, 95%) was added to a solution of tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (1400 g, 2.231 mol) in THE (14 L) and water (80 mL) at r.t. The mixture was heated to 50° C. for 1-3 h to complete the reaction. The mixture was cooled to r.t. and acidified with aq. HCl (1 M) to about pH 5. THE was removed under vacuum. The product was extracted by DCM (6 L). The aqueous phase was separated and extracted with DCM (6 L). The combined organic phases were concentrated under vacuum to give the product in DCM solution (6 L). The concentrated DCM solution was added to MTBE (7 L) was added to the residue and the mixture slurry was agitated at r.t. for 2 h. n-Heptane (7 L) was added to the mixture. The DCM was removed under vacuum. The solids were isolated by filtration and the wet cake was washed with n-heptane (2×3 L). The solids were dried under vacuum at about 50° C. to give the title compound that was used for next step without further purification.

The alternative atropisomer (Sₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid is prepared by an analogous route by performing processes analogous to Steps 14 and 15b starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 16. tert-Butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

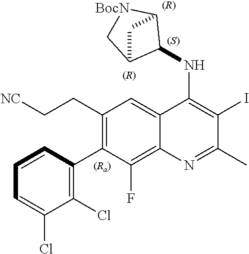

To a mixture of (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (132 g, 220 mmol), and sodium phosphate (74.4 g, 440 mmol) in anhydrous MeCN (1614 mL) was added N-iodosuccinimide (94 g, 396 mmol) and the mixture was stirred for 1 h. Water (1.6 L) was added to the mixture and resulting slurry was stirred for 5 h at r.t. The solids were isolated by filtration and the wet cake was reslurried in water (2.6 L) at r.t. for 5 h. The solids were isolated by filtration and the wet cake was washed with water (2×250 mL). The solids were dried under vacuum at about 50° C. to give the title compound (120 g, 80% yield). LCMS calc. for C₃₉H₂₈Cl₂FIN₄O₂: 680.06; Found: 681 (M+H⁺). ¹H-NMR (400 MHz, DMSO-d₆) δ 7.94 (s, 1H), 7.82 (dd, J=8.0, 1.6 Hz, 1H), 7.56 (t, J=7.8 Hz, 1H), 7.50 (dd, J=7.7, 1.6 Hz, 1H), 5.49 (s, 1H), 4.28 (s, 2H), 3.09 (s, 1H), 2.96-2.58 (m, 8H), 1.71 (s, 1H), 1.59-0.96 (m, 11H).

The alternative atropisomer tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate is prepared by an analogous route by performing processes analogous to Steps 14-16 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 17. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate

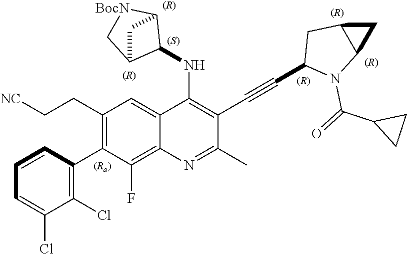

A mixture of cyclopropyl((1R,5R)-3-ethynyl-2-azabicyclo[3.1.0]hexan-2-yl)methanone (47.5 g, 260 mmol), tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (136.5 g, 200 mmol), and tetrabutylammonium acetate (242 g, 801 mmol) in DMF (1100 mL) was subsurface purged with nitrogen gas for 10 min. Tris(dibenzylideneacetone)dipalladium(0) (2.75 g, 3 mmol) was added to the mixture. The mixture was subsurface purged with nitrogen gas for another 15 min. before heating at 70° C. for 1 h. The reaction mixture was cooled to r.t. and added to half saturated aq. NaHCO₃ (2200 mL). The solids were isolated by filtration and the wet cake was washed with water (600 mL). The solids were dried under vacuum at about 50° C. and purified by silica gel column eluted with 0-2% MeOH in EtOAc to give the title compound (142 g, 96% yield). ¹H NMR (400 MHz, DMSO-d₆) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

The alternative atropisomer tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate is prepared by an analogous route by performing processes analogous to Steps 14-17 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 17a. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate

The title compound can alternatively be prepared by the following method. A mixture of cyclopropyl((1R,5R)-3-ethynyl-2-azabicyclo[3.1.0]hexan-2-yl)methanone (17.7 kg, 101 mol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (64.7 kg, 95 mol), copper (I) iodide (0.42 kg 2 mol), tris (4-fluorophenyl)phosphine (0.39 kg, 1 mol) and K₂CO₃ (36.4 kg, 191 mol) in DMSO (488.4 L) was subsurface purged with nitrogen gas for 30 min. Palladium (II) acetate (60 g, 30 mmol) was added to the mixture. The mixture was subsurface purged with nitrogen gas for another 30 min. before heating to 50° C. for more than 10 h. The reaction mixture was cooled to r.t. and EtOAc (906 L) was added, followed by slow addition of water (1267 L) was added. The mixture was stirred at r.t. for 30 min. and filtered over a diatomaceous earth bed. The diatomaceous earth bed was rinsed with EtOAc (33 L). The organic phase was separated from the aqueous phase and the aqueous phase was back extracted with EtOAc (195 L). The combined organic phase was washed with water (195 L). To the EtOAc phase was added water (130 L) and ammonium pyrrolidinedithiocarbamate (3.1 kg, 19 mol). The mixture was agitated at 50° C. for no less than 4 h. The mixture was cooled to r.t. and polish filtered. The aqueous phase was separated and discarded. The organic phase was washed with water (325 L). The organic phase was heated to 50° C. and passed through activated carbon cartridge. The solution was concentrated under vacuum and solvent swapped into toluene to remove residual water to give desired product in 98% solution yield. The toluene solution was solvent swapped into NMP for next step indole-cyclization without further purification. ¹H NMR (400 MHz, DMSO-d₆) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

The alternative atropisomer tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate is prepared by an analogous route by performing processes analogous to Steps 14-17 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 18. tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (Compound 2)

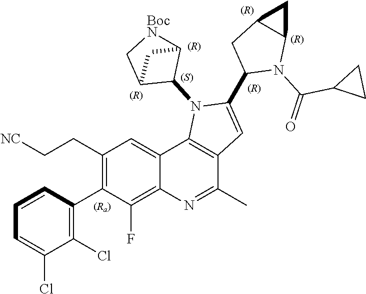

To a mixture of tert-butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate (141.0 g, 159 mmol) and Cs₂CO₃ (78 g, 238 mmol) in DMSO (1 L) or NMP was heated at 80-85° C. for 1.5 h. The reaction mixture was cooled to r.t. and water (2 L) was gradually added. The product was gradually precipitated out of the solution. The resulting slurry was stirred at r.t. for 1 h. The solids were isolated by filtration and the wet cake was washed with water (2×300 mL). The wet solids were dried under vacuum. The solids were purified by flash chromatography with 60-100% EtOAc in DCM. The solvents were removed and the solids in heptane (840 mL) were crystallized from EtOAc (420 mL) and MTBE (420 mL) and heptane (840 mL) to give the title compound (122 g, 87% yield). LCMS calc. for C₄₀H₄₀Cl₂FN₅O₃: 727.25; Found: 728 (M+H⁺). ¹H NMR (500 MHz, DMSO-d₆) δ 8.12 (s, 1H), 7.81 (dt, J=8.0, 2.1 Hz, 1H), 7.55 (td, J=7.8, 5.0 Hz, 1H), 7.45-7.29 (m, 1H), 6.26 (s, 1H), 5.81-5.49 (m, 1H), 5.34-5.13 (m, 1H), 5.00 (dd, J=14.3, 6.8 Hz, 1H), 4.19-3.97 (m, 1H), 3.63 (dt, J=6.8, 3.1 Hz, 1H), 3.40 (d, J=9.4 Hz, 1H), 3.27-3.09 (m, 1H), 2.95 (dt, J=14.2, 7.6 Hz, 1H), 2.89-2.73 (m, 3H), 2.70 (d, J=2.7 Hz, 4H), 2.34-2.20 (m, 1H), 2.21-1.97 (m, 2H), 1.73 (dp, J=15.0, 4.8 Hz, 1H), 1.66-1.34 (m, 2H), 1.21-1.03 (m, 1H), 1.02-0.79 (m, 4H), 0.78-0.22 (m, 11H).

The alternative atropisomer tert-butyl (1R,4R,5S)-5-((Sₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate is prepared by an analogous route by performing processes analogous to Steps 14-18 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 19. 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile

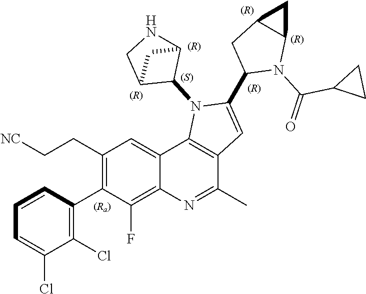

To a solution of tert-butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (167.7 g, 230.1 mmol) in DCM (1.35 L) was added TMSI (69 g, 345 mmol) at r.t. and the mixture was stirred for 1 h. Aq. NaHCO₃ (500 mL) was added to quench the reaction. The organic phase was isolated and washed with water. The solvent was evaporated by rotary evaporation, and the residue was passed over silica gel bed (1-20% MeOH in DCM). The solvent was swapped into EtOAc and MTBE to give crystalline product (136 g, 94% yield). LCMS calc. for C₃₅H₃₂Cl₂FN₅O: 627.20; Found: 628 (M+H⁺). ¹H-NMR (400 MHz, DMSO-d₆) δ ¹H NMR (500 MHz, DMSO-d₆) δ 8.15 (d, J=13.6 Hz, 1H), 7.89-7.73 (m, 1H), 7.64-7.33 (m, 2H), 6.69-6.14 (m, 1H), 5.76-5.43 (m, 1H), 4.97 (d, J=4.9 Hz, 1H), 4.31 (dd, J=17.0, 6.0 Hz, 1H), 4.18-3.94 (m, 1H), 3.58-3.45 (m, 1H), 2.94 (dt, 2H, J=12.4, 6.1 Hz), 2.89-2.56 (m, 8H), 2.44-2.19 (m, 2H), 2.07 (d, J=12.9 Hz, 1H), 1.96-1.54 (m, 3H), 1.30-1.13 (m, 1H), 1.06-0.20 (m, 6H).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is prepared by an analogous route by performing processes analogous to Steps 14-19 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Example 2. 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate

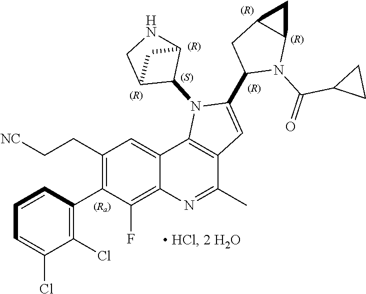

To a solution of dissolved 3-((Rₐ)-1-((1R,4R,5S)-2-azabicyclo[2.1.1]-hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile free base (53.8 g, 85 mmol) in MeOH (110 mL), EtOAc (50 mL), water (11 mL) and MTBE (110 mL) was added aq. HCl (6 M, 14.5 mL) at 30-50° C. The mixture was seeded, and the solution gradually turned cloudy. MTBE (440 mL) was slowly added to the mixture at about 40° C. over 1 h. The mixture was cooled to about 15° C. and agitated for 2 h. The solids were isolated by filtration and the wet cake was washed with 5% MeOH and 20% EtOAc in MTBE (2×110 mL). The wet solids were slurried in EtOAc (270 mL) and dried under vacuum at about 50° C. to give the title compound (53.7 g, 90% yield). LCMS calc. for C₃₅H₃₂Cl₂FN₅O: 627.20; Found: 628 (M+H⁺). ¹H NMR (500 MHz, DMSO-d₆) δ 8.15 (s, 1H), 7.83 (dd, J=8.1, 1.6 Hz, 1H); 7.57 (dd, J=7.9, 7.9, 1H); 7.45 (dd, J=7.7, 1.6 Hz, 1H); 6.44 (s, 1H); 5.65 (s, 1H); 5.51 (d, J=10.6 Hz, 1H); 4.14 (td, J=6.4, 2.6 Hz, 1H); 3.84-3.90 (m, 1H); 3.30-3.37 (m, 1H); 3.43-3.50 (m, 1H); 2.86-2.95 (m, 1H); 2.83-2.92 (m, 1H); 2.79 (s, 3H); 2.70-2.79 (m, 1H); 2.29-2.35 (m, 1H); 2.25-2.32 (m, 1H); 1.97 (dd, J=13.0, 2.6 Hz, 1H); 1.69-1.83 (m, 1H); 1.65 (d, J=9.1 Hz, 1H); 0.91-1.00 (m, 2H); 0.82-0.88 (m, 2H); 0.72-0.80 (m, 1H); 0.63-0.69 (m, 1H). ¹³C NMR (125 MHz, DMSO-d₆) δ 171.6; 145.8; 132.8; 135.1; 132.8; 131.9; 131.5; 131.4; 129.2; 101.6; 120.7; 57.9; 56.5; 44.5; 42.5; 30.5; 38.3; 32.8; 22.1; 17.5; 17.1; 13.2; 13.0; 7.70; 7.80. ¹⁹F NMR (376 MHz, DMSO-d₆) δ −122.1 (s).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate is prepared by an analogous route starting from 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile instead of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

### Example 3. Alternative Synthesis for Preparing ethyl 4-chloro-6-(2-cyanoethyl)-7-(2,3-dichlorophenyl)-8-fluoro-2-methylquinoline-3-carboxylate (step 13 of Example 1)

### Step 1. Methyl 6-(2-(1,3-dioxolan-2-yl)ethyl)-3-amino-2′,3′-dichloro-2-fluoro-[1,1′-biphenyl]-4-carboxylate

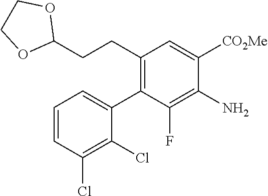 [SMILES: COC(=O)c1cc(CCC2OCCO2)c(-c2cccc(Cl)c2Cl)c(F)c1N]

To a round bottom flask under nitrogen was charged 2-vinyl-1,3-dioxolane (6.52 g, 65.1 mmol, 1.05 eq.). 0.5 M 9-BBN dimer in THE (149 mL, 74.4 mmol, 1.20 eq.) solution was cannulated to the reaction flask over 15 min. at r.t. The reaction mixture was then heated to 40° C. and stirred for NLT 1 h. After fully consumption of the 2-vinyl-1,3-dioxolane, methyl 3-amino-6-bromo-2′,3′-dichloro-2-fluoro-[1,1′-biphenyl]-4-carboxylate (25.0 g, 62.0 mmol, 1.0 eq.) was added, followed by Bis(di-tert-butyl(4-dimethylaminophenyl)phosphine)dichloropalladium(II) (0.088 g, 0.124 mmol, 2 mol %), potassium phosphate, tribasic (26.3 g, 124 mmol, 2.0 eq.), and water (25 mL, 1 V). The resulting reaction mixture was sparged by nitrogen for another 15 min. and heated to 60° C. The reaction was completed after stirring at 60° C. for 16 h. Upon completion, the reaction was quenched by 1 M aq. HCl solution until pH=7. The reaction mixture was then diluted by EtOAc (50 mL, 2 V) and the organic layer was separated. The aqueous layer was washed by another portion of EtOAc (25 mL, 1 V) and the organic layer was separated. The combined organic layer was dried on Na₂SO₄ and filtrated through a pad of diatomaceous earth. The filtrate was collected and the solvent was evaporated in vacuo. The crude product was re-dissolved MeCN (150 mL, 6 V) at 60° C. and slowly added water (100 mL, 4 V) to crush out solid products. The solid slurry was stirred at 60° C. for 2 h then slowly cooled to r.t. to stir for an additional 2 h before isolation. The reaction mixture was filtrated and solids were collected. The wet solid cake was washed by 4 V of 30% v/v MeCN in water. After drying the solids by pulling air through for overnight, the desired product was obtained as light grey solids (19.2 g, yield=75%). LCMS calc. for C₁₉H₁₈Cl₂FNO₄: 414.25, Found: 415 (M+H). ¹H NMR (400 MHz, DMSO-d6) δ 7.75 (dd, J=8.1, 1.5 Hz, 1H), 7.54 (d, J=1.4 Hz, 1H), 7.49 (t, J=7.9 Hz, 1H), 7.36 (dd, J=7.7, 1.6 Hz, 1H), 6.48 (s, 2H), 4.60 (t, J=4.8 Hz, 1H), 3.85 (s, 3H), 3.80-3.59 (m, 4H), 2.33 (ddd, J=14.3, 9.4, 6.9 Hz, 1H), 2.22 (ddd, J=14.3, 9.5, 6.8 Hz, 1H), 1.56 (ttd, J=9.6, 7.0, 4.7 Hz, 2H) ppm.

### Step 2. 6-(2-(1,3-Dioxolan-2-yl)ethyl)-3-amino-2′,3′-dichloro-2-fluoro-[1,1′-biphenyl]-4-carboxylic acid

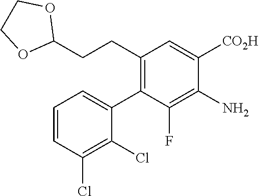 [SMILES: Nc1c(C(=O)O)cc(CCC2OCCO2)c(-c2cccc(Cl)c2Cl)c1F]

Methyl 6-(2-(1,3-dioxolan-2-yl)ethyl)-3-amino-2′,3′-dichloro-2-fluoro-[1,1′-biphenyl]-4-carboxylate (19.2 g, 46.3 mmol, 1.0 eq.) was dissolved in a mixture of THE (80 mL) and MeOH (20 mL). To the reaction mixture was added 1 M aq. NaOH solution (93 mL, 2.0 eq.) and heated to 50° C. for 3 h. Upon completion, the reaction was cooled to r.t. and slowly acidified by 1 M aq. HCl solution to pH=3-4. The resulting solid slurry was stirred at r.t. for NLT 1 h. The solid was isolated, and the wet cake was washed by 20% v/v MeCN in water. After drying the solids by pulling air through for overnight, the desired product was obtained as white solids (18.5 g, yield=99%). LCMS calc. for C₁₈H₁₆Cl₂FNO₄: 400.23; Found: 401 (M+H). ¹H NMR (400 MHz, DMSO-d₆) δ 7.75 (dd, J=8.0, 1.5 Hz, 1H), 7.53 (d, J=1.4 Hz, 1H), 7.49 (t, J=7.9 Hz, 1H), 7.36 (dd, J=7.7, 1.6 Hz, 1H), 4.60 (t, J=4.8 Hz, 1H), 3.79-3.60 (m, 4H), 3.31 (s, 2H), 3.28 (s, 1H), 2.32 (ddd, J=14.5, 9.4, 6.9 Hz, 1H), 2.21 (ddd, J=14.3, 9.5, 6.8 Hz, 1H), 1.56 (dddd, J=9.4, 7.0, 4.8, 2.4 Hz, 2H) ppm.

### Step 3. Ethyl 6-(2-(1,3-dioxolan-2-yl)ethyl)-7-(2,3-dichlorophenyl)-8-fluoro-2-methyl-4-oxo-1,4-dihydroquinoline-3-carboxylate

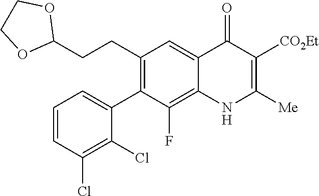 [SMILES: CCOC(=O)c1c(C)[nH]c2c(F)c(-c3cccc(Cl)c3Cl)c(CCC3OCCO3)cc2c1=O]

To a reaction round bottom flask purged by nitrogen was dissolved 6-(2-(1,3-dioxolan-2-yl)ethyl)-7-(2,3-dichlorophenyl)-8-fluoro-2H-benzo[d][1,3]oxazine-2,4(1H)-dione (18.6 g, 43.6 mmol, 1.0 eq.) and sodium (Z)-4-ethoxy-4-oxobut-2-en-2-olate (13.28 g, 87.0 mmol, 2.0 eq.) in DMSO (100 mL, 5 V). The reaction was stirred at r.t. for 15 min. until fully dissolved. The reaction was then heated to 60-65° C. for 16 h followed by an additional 2 h at 90° C. Upon completion, the reaction was cooled to r.t. and diluted by water (100 mL, 5 V). The resulting reaction mixture was acidified by aqeuous HCl solution until pH=7. The solid slurry was re-heated to 45-50° C. for 2 h and cooled to r.t. After stirring at r.t. overnight, the crude product was isolated. The crude product was re-slurried in 1:1 v/v MTBE in hexane for 2 h at r.t. The solid was then isolated. The wet cake was washed by hexane and dried overnight by pulling air through. The desired product was obtained as white solids (17.8 g, yield=83%). LCMS calc. for C₂₄H₂₂Cl₂FNO₅: 494.34; Found: 495 (M+H). ¹H NMR (400 MHz, DMSO-d6) δ 11.82 (s, 1H), 7.86 (s, 1H), 7.82 (dd, J=8.0, 1.6 Hz, 1H), 7.56 (t, J=7.8 Hz, 1H), 7.48 (dd, J=7.7, 1.6 Hz, 1H), 4.66 (t, J=4.7 Hz, 1H), 4.27 (q, J=7.1 Hz, 2H), 3.86-3.58 (m, 4H), 2.56 (d, J=7.9 Hz, 1H), 2.44 (s, 4H), 1.74-1.57 (m, 2H), 1.29 (t, J=7.1 Hz, 3H).

### Step 4. Methyl 3-amino-2′,3′-dichloro-2-fluoro-6-(3-oxopropyl)-[1,1′-biphenyl]-4-carboxylate

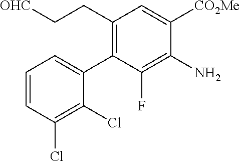 [SMILES: COC(=O)c1cc(CCC=O)c(-c2cccc(Cl)c2Cl)c(F)c1N]

To a solution of methyl 6-(2-(1,3-dioxolan-2-yl)ethyl)-3-amino-2′,3′-dichloro-2-fluoro-[1,1′-biphenyl]-4-carboxylate (6.5 g, 15.7 mmol) in 1,4-dioxane (65 mL) was added 1 M HCl (110 mL, 110 mmol). The mixture was heated to 70-75° C. for 2 h. The mixture was cooled to r.t. and MTBE (30 mL) was added. The organic phase was separated, and the aqueous phase was back extracted with MTBE (3×30 mL). The combined organic phase was washed with water (2×30 mL). The organic phase was concentrated to give desired product (6.14 g, 88%). LCMS calc. for C₁₇H₁₄Cl₂FNO₃: 369.03; Found: 370 (M+H). ¹H NMR (400 MHz, DMSO-d₆) δ 9.54 (d, J=1.1 Hz, 1H), 7.76 (dd, J=8.1, 1.5 Hz, 1H), 7.59-7.45 (m, 2H), 7.39 (dd, J=7.7, 1.5 Hz, 1H), 6.51 (s, 3H), 3.86 (s, 4H), 2.68-2.52 (m, 2H), 2.50-2.34 (m, 3H).

### Step 5. Methyl 3-amino-2′,3′-dichloro-6-(2-cyanoethyl)-2-fluoro-[1,1′-biphenyl]-4-carboxylate

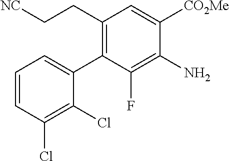 [SMILES: COC(=O)c1cc(CCC#N)c(-c2cccc(Cl)c2Cl)c(F)c1N]

To a mixture of methyl 3-amino-2′,3′-dichloro-2-fluoro-6-(3-oxopropyl)-[1,1′-biphenyl]-4-carboxylate (5.2 g, 11.6 mmol), hydroxylamine hydrochloride (0.9 g, 12.8 mmol) and triethylamine (1.3 g, 12.8 mmol) in DMF (51 mL) was added propane phosphonic acid anhydride (10.8 mL, 15.3 mmol) and stirred for 3 h. The reaction mixture is diluted with water (100 mL) and extracted with MTBE (3×100 mL). The combined organic phase was washed with water (2×50 mL) and dried over sodium sulfate. The organic solvent was removed by rotary evaporation and the residue was purified by silica gel column (0-10% methanol in DCM) to give desired product (1.7 g, 40%). LCMS calc. for Chemical Formula: C₁₇H₁₃Cl₂FN₂O₂: 366.03; Found: 367 (M+1). ¹H NMR (400 MHz, DMSO-d₆) δ 7.78 (dd, J=8.1, 1.5 Hz, 1H), 7.68 (d, J=1.5 Hz, 1H), 7.52 (t, J=7.9 Hz, 1H), 7.42 (dd, J=7.6, 1.6 Hz, 1H), 6.61 (s, 2H), 3.87 (s, 3H), 2.67-2.51 (m, 2H), 2.47-2.35 (m, 1H).

### Step 6. Ethyl 4-chloro-6-(2-cyanoethyl)-7-(2,3-dichlorophenyl)-8-fluoro-2-methylquinoline-3-carboxylate

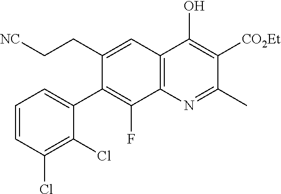 [SMILES: CCOC(=O)c1c(C)nc2c(F)c(-c3cccc(Cl)c3Cl)c(CCC#N)cc2c1O]

A mixture of methyl 3-amino-2′,3′-dichloro-6-(2-cyanoethyl)-2-fluoro-[1,1′-biphenyl]-4-carboxylate (10.00 g, 27.0 mmol) ethyl (Z)-3-ethoxybut-2-enoate (5.3 g, 32.4 mmol) and pyridinium p-toluenesulfonate (0.14 g, 0.54 mmol) in anhydrous toluene (100 mL) was heated at reflux (110° C.) for 21 h. The mixture was cooled to r.t., 2.68 M sodium ethoxide in ethanol (15.1 mL, 40.5 mmol) was then added. The resulting orange solution was heated at 80° C. for 4 h. The reaction mixture was cooled to r.t., diluted with water (100 mL), acidified with 1 M HCl (42 mL) to pH 5. EtOAc (500 mL) was added to the reaction mixture. The organic layer was separated, dried over sodium sulfate, filtered, and concentrated by rotary evaporation. The residue was purified by silica gel column and eluting with 0-70% EtOAc/DCM to give the desired product as a light-yellow solid (7.2 g, 59% yield). LCMS calc. for C₂₂H₁₇Cl₂FN₂O₂: 446.03; Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Example 4. Synthesis of cyclopropyl((1R,3R,5R)-3-ethynyl-2-azabicyclo[3.1.0]hexan-2-yl)methanone (Step 17 in Example 1)

### Step 1. 1-(tert-Butyl) 2-ethyl (R)-2,3-dihydro-1H-pyrrole-1,2-dicarboxylate

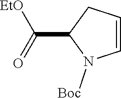 [SMILES: CCOC(=O)[C@H]1CC=CN1C(=O)OC(C)(C)C]

To a solution of 1-(tert-butyl) 2-ethyl (R)-5-oxopyrrolidine-1,2-dicarboxylate (241 g, 0.938 mol) in anhydrous toluene (1.6 L) was added 1 M lithium triethyl borohydride in tetrahydrofuran (1.01 L, 1.01 mol) dropwise at −50-−40° C. over 1 h. After addition, the mixture was stirred for 1 h at about −50° C. DIPEA (726 mL, 4.17 mol) was added to the mixture dropwise over 1 h. 4-Dimethylaminopyridine (1.49 g, 12.2 mmol, 0.013 eq.) was added to the mixture, followed by the dropwise addition of trifluoroacetic anhydride (156.5 mL, 1.126 mol) over 1.5 h. After addition, the mixture was stirred for 1 h at about −50° C., then slowly warmed to r.t. The mixture was stirred for 1 h at r.t. The reaction mixture was cooled to 0° C. and diluted slowly with water (2.41 L), while maintaining the temperature below 10° C. during addition. The organic layer was separated and washed with water (2.41 L) and saturated brine (720 mL). The organic layer was dried over sodium sulfate (120 g). The solution was concentrated under reduced pressure to give desired product (230 g, quant.) as yellow oil. GCMS calc. for C₁₂H₁₉NO₄: 241.1; Found: 214.2 (M⁺). ¹H-NMR (400 MHz, CDCl₃) δ 6.70-6.48 (m, 1H), 4.99-4.86 (m, 1H), 4.70-4.52 (m, 1H), 4.30-4.11 (m, 2H), 3.15-2.98 (m, 1H), 2.73-2.57 (m, 1H), 1.53-1.38 (m, 9H), 1.34-1.21 (m, 4H).

### Step 2. 2-(tert-Butyl) 3-ethyl (1R,3R,5R)-2-azabicyclo[3.1.0]hexane-2,3-dicarboxylate

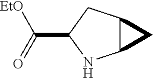

To a solution 1-(tert-butyl) 2-ethyl (R)-2,3-dihydro-1H-pyrrole-1,2-dicarboxylate (230 g, 0.938 mol) in toluene (2.3 L) was added 1.1 M diethylzinc in toluene (1.7 L, 1.87 mol) at −30 to −25° C. over 1 h. Chloroiodomethane (273 mL, 3.752 mol) was added to the mixture dropwise over 2 h at about −30 to −20° C. and the mixture was stirred for 16 h. Half-saturated sodium bicarbonate (2.3 L) was added to the mixture and the mixture was warmed up to r.t. The mixture was filtered over diatomaceous earth to remove white solids and the filter bed was rinsed with toluene (1.5 L). The organic layer was separated from the filtrate and washed with water (2×1.15 L) and saturated brine (1.15 L). The toluene solution was concentrated under reduced pressure to give a 6 to 1 mixture (231 g) of 2-(tert-Butyl) 3-ethyl (1R,3R,5R)-2-azabicyclo[3.1.0]hexane-2,3-dicarboxylate and 2-(tert-butyl) 3-ethyl (1S,3R,5S)-2-azabicyclo[3.1.0]hexane-2,3-dicarboxylate as yellow oil as determined by GCMS analysis.

Aqueous methyl amine (40%, 344 g) was added to a crude mixture product obtained above (226 g) and the mixture was stirred for 16 h at r.t. Water (340 mL) and methyl tert-butyl ether (340 mL) was added to the mixture. The organic layer was separated and washed with water (340 mL) and saturated brine (230 mL). The solution was concentrated under reduced pressure to give 2-(tert-butyl) 3-ethyl (1R,3R,5R)-2-azabicyclo[3.1.0]hexane-2,3-dicarboxylate (177 g, 73% calc. yield) as yellow oil, which contained 2% 2-(tert-butyl) 3-ethyl (1S,3R,5S)-2-azabicyclo[3.1.0]hexane-2,3-dicarboxylate as determined by GCMS analysis. GCMS calc. for C₁₃H₂₁NO₄: 255.1; Found: 255.1 (M⁺). ¹H-NMR (400 MHz, CDCl₃) δ 4.56-4.39 (m, 1H), 4.18-4.01 (m, 2H), 3.51-3.36 (m, 1H), 2.60-2.42 (m, 1H), 2.00-1.92 (m, 1H), 1.45-1.32 (m, 9H), 1.23-1.15 (m, 4H), 0.87-0.79 (m, 1H), 0.70-0.56 (m, 1H).

### Step 3. tert-Butyl (1R,3R,5R)-3-(hydroxymethyl)-2-azabicyclo[3.1.0]hexane-2-carboxylate

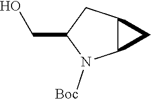

A solution of 2-(tert-butyl) 3-ethyl (1R,3R,5R)-2-azabicyclo[3.1.0]hexane-2,3-dicarboxylate (177 g, 0.694 mol) in tetrahydrofuran (1.56 L) was added to 1 M lithium aluminum hydride solution in tetrahydrofuran (777 mL, 0.777 mol, 1.12 eq.) at about 0-10° C. over 1 h. After addition, the mixture was stirred for 2 h at 3° C. Water (27 mL) was added to the mixture dropwise to quench the reaction. Sodium hydroxide solution (15%, 27 mL) and water (80 mL) were sequentially added to the mixture dropwise. The mixture was stirred at r.t. for 1 h. DCM (2.35 L) was added to the mixture. The suspension was filtered through diatomaceous earth (100 g) bed and rinsed with DCM (300 mL). The filtrate was concentrated under reduced pressure and dried under vacuum oven at 40° C. for 18 h to give tert-butyl (1R,3R,5R)-3-(hydroxymethyl)-2-azabicyclo[3.1.0]hexane-2-carboxylate (133 g, 90% yield) as yellow oil which contained 2% of an isomer as determined by GCMS analysis. GCMS calc. for C₁₁H₁₉NO₃: 213.1; Found: 213.2 (M⁺). ¹H-NMR (400 MHz, CDCl₃) δ 4.83 (brs, 1H), 4.34 (brs, 1H), 2.45 (ddd, 1H), 1.55-1.43 (m, 12H), 0.80 (q, 1H), 0.40 (brs, 1H).

### Step 4. tert-Butyl (1R,3R,5R)-3-formyl-2-azabicyclo[3.1.0]hexane-2-carboxylate

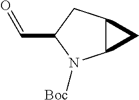

DMSO (42.7 mL, 0.603 mol) was added to oxalyl chloride (26.4 mL, 0.301 mol) in DCM (535 mL) dropwise at −78° C. over 30 min., while maintaining the temperature below −60° C. during addition. After stirring at −78° C. for 30 min. tert-butyl (1R,3R,5R)-3-(hydroxymethyl)-2-azabicyclo[3.1.0]hexane-2-carboxylate (53.5 g, 0.251 mol) in DCM (535 mL) was added to solution dropwise at −78° C. over 40 min. After stirring at −78° C. for 30 min., NEt₃ (104.9 mL, 0.753 mol) was added to solution dropwise at −78° C. over 40 min. After stirring at −78° C. for 1 h, the reaction mixture was warmed to 0° C. and stirred for 30 min. Water (888 mL) was added to the mixture and stirred for 20 min. The aqueous layer was separated and extracted with DCM (2×888 mL). The combined organic layers were sequentially washed with 1 M HCl (888 mL), water (888 mL) and saturated brine (888 mL). The organic layer was concentrated under reduced pressure to give tert-butyl (1R,3R,5R)-3-formyl-2-azabicyclo[3.1.0]hexane-2-carboxylate (44 g, 83% yield) as yellow oil. GCMS calc. for C₁₁H₁₇NO₃: 213.1; Found: 213.2 (M⁺). ¹H-NMR (400 MHz, CDCl₃) δ 9.54-9.31 (m, 1H), 4.64-4.39 (m, 1H), 3.68-3.45 (m, 1H), 2.68-2.33 (m, 1H), 2.24-2.10 (m, 1H), 1.53-1.41 (m, 10H), 0.88-0.71 (m, 1H), 0.39-0.28 (m, 1H).

### Step 5. tert-Butyl (1R,3R,5R)-3-ethynyl-2-azabicyclo[3.1.0]hexane-2-carboxylate

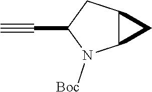

K₂CO₃ (28.8 g, 0.209 mol, 2 eq.) was added to a solution of tert-butyl (1R,3R,5R)-3-formyl-2-azabicyclo[3.1.0]hexane-2-carboxylate (22 g, 0.104 mol) in methanol (352 mL) at 0-5° C. Dimethyl (1-diazo-2-oxopropyl)phosphonate (18.3 mL, 0.110 mol) was added to the mixture dropwise at 0-5° C. for 30 min., while maintaining the temperature at <5° C. during addition. After stirring at 0-5° C. for 15 min., the reaction mixture was warmed up to r.t. and stirred for 2 h. Water (372 mL) and EtOAc (930 mL) was added to the mixture, which was stirred for 15 min. The aqueous layer was separated and extracted with EtOAc (372 mL). The combined organic layers were washed with water (560 mL) and saturated brine (560 mL). The organic solution was concentrated under reduced pressure and purified over silica gel and eluted with a gradient of 0-10% EtOAc in heptane to give a 7 to 1 mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 74% calc. yield) as light yellow oil. GCMS calc. for C₁₂H₁₇NO₂: 207.1; Found: 207.0 (M⁺). ¹H-NMR (400 MHz, CDCl₃) δ 4.78-4.54 (m, 1H), 3.60-3.46 (m, 1H), 2.52-2.40 (m, 1H), 2.30-2.22 (m, 1H), 2.18-2.08 (m, 1H), 1.50-1.48 (m, 9H), 1.16-1.05 (m, 1H), 0.91-0.80 (m, 1H), 0.78-0.66 (m, 1H).

### Step 6. Cyclopropyl((1R,3R,5R)-3-ethynyl-2-azabicyclo[3.1.0]hexan-2-yl)methanone

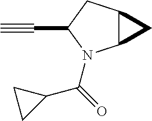

A mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 0.39 mol) and 4M HCl in dioxane (297 mL, 1.19 mol, 3 eq.) was stirred at rt for 4 h. The reaction mixture was diluted with THE (1.23 L) and cooled to 0° C. NEt₃ (275.8 mL, 1.98 mol) was added to the reaction at 0° C. dropwise over 1.5 h while maintaining the temperature at <10° C. during addition. Cyclopropanecarbonyl chloride (45.4 g, 0.43 mol) was added to the reaction at 0° C. The reaction was warmed to r.t. and stirred for 3 h. 1 M HCl (410 mL, 5 vol) and DCM (820 mL) was added. The aqueous layer was separated and extracted with DCM (2×820 mL). The combined organic layers were washed with water (820 mL) and saturated brine (820 mL). The organic layer was concentrated under reduced pressure to give a crude residue (60 g). Diatomaceous earth (120 g) was added to the crude residue and the mixture was dried under reduced pressure to give a dried load powder (186 g). The dried load powder was purified on a silica gel column (1.5 kg) and eluted with a gradient of 15 to 40% EtOAc in heptane. The desired fractions were concentrated under reduced pressure and dried under vacuum at 30° C. for 18 h to give the title compound (40.8 g, 59% yield) as brown oil. GCMS calc. for C₁₂H₁₇NO₂: 175.1; Found: 175.0 (M⁺). ¹H-NMR (400 MHz, DMSOd₆) δ 5.14 (dt, 0.45H), 4.81 (dt, 0.55H), 3.82 (t, 0.55H), 3.71 (t, 0.45H), 3.42 (d, 0.45H), 3.15 (d, 0.55H), 2.57 (ddd, 0.45H), 2.44 (ddd, 0.55H), 2.09 (dd, 0.45H), 2.04 (ddd, 0.55H), 1.97 (dd, 0.55H), 1.86-1.69 (m, 1H), 1.62 (dddd, 0.45H), 1.01 (td, 0.55H), 0.90 (td, 0.45H), 0.87-0.68 (m, 5H).

### Example 5. Synthesis of tert-Butyl (1R,4R,5S)-5-amino-2-azabicyclo[2.1.1]hexane-2-carboxylate oxalate (Step 14a in Example 1)

### Step 1. (E)-4-Methoxybut-3-en-2-one

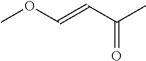

A mixture of 4,4-dimethoxy-2-butanone (350 g, 1.0 eq) and sodium acetate (11 g, 0.05 eq.) was heated to 145-150° C. under nitrogen atmosphere and the resulting methanol is purged during the heating process. When the reaction is complete, the mixture was cooled to 70-80° C. The product was distilled under vacuum to give desired product (130 g, yield 50%). 1H NMR (CD₂Cl₂/CHDOD, 400 MHz): δ 7.60 (d, 1H, J=12.8 Hz) 5.53 (d, 1H, J=12.8 Hz), 3.81 (s, 3H), 2.17 (s, 3H). 13C NMR (CD₂Cl₂/CD₃OD, 100.6 MHz): δ 27.1, 58.0, 107.0, 165.2, 199.6.

### Step 2. (E)-4-(Allylamino)but-3-en-2-one

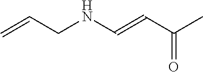

A mixture of (E)-4-methoxybut-3-en-2-one (150 g) and NEt₃ (182 g) in DCM (450 mL) was added was agitated under nitrogen at 10-15° C. Allylamine hydrochloride aqueous solution (60%, 234 g) is slowly added to the mixture at 10-15° C. After the addition, the mixture is agitated for 30 min. When the reaction was completed, water (150 g) was added to the reaction mixture. The organic phase was separated, and the water phase was extracted with DCM (300 mL). The combined organic phases were washed with brine (150 mL) and organic phase was concentrated under vacuum to give crude product as yellow oil (175 g, yield 93%). 1H NMR (500 MHz, CDCl₃): δ 9.75 (bs, 1H); 6.58 (dd, 1H, J=16.8, 2); 5.78-5.86 (m, 1H); 5.19 (d, 1H, J=16.8)); 5.14 (d, 1H, J=10, 1)); 5.00 (d, 1H, J=10, 1); 3.74-3.77 (m, 2H); 2.03, (s, 3H). 13C NMR (125 Hz, CDCl3): 197.5; 153.2; 165.3; 117.6; 94.9; 51.1; 29.2.

### Step 3. tert-Butyl (E)-allyl(3-oxobut-1-en-1-yl)carbamate

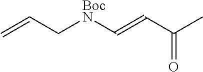 [SMILES: C=CCN(/C=C/C(C)=O)C(=O)OC(C)(C)C]

A mixture of (E)-4-(allylamino)but-3-en-2-one (130 g), trimethylamine (105 g), N,N-dimethylaminopyridine (13 g) in toluene (390 mL) was heated to 50-55° C. (Boc)₂O (259 g) was added in portion while maintained the reaction temperature between 50-55° C. After the reaction mixture was agitated for 2 h at 50-55° C. to complete the reaction. The mixture was cooled to 10-15° C. and 3 M aq. HCl was added to the mixture until the pH 5-6. The organic phase was separated, and the aqueous phase was extracted with toluene (260 mL). The combined organic phases were washed with water (260 mL). Activated charcoal (1 g) was added. The mixture was agitated at 50-55° C. for 1 h before cooling the mixture to 20-30° C. The mixture was filtered over diatomaceous earth bed and the diatomaceous earth bed was rinsed with toluene. The filtrated was concentrated to a residue and the residue was coevaporated with MeCN to give a residue as yellow oil (189 g, 80% yield). 1H NMR (500 MHz, CDCl₃): δ 8.11 (d, 1H, J=15); 5.68-5.73 (m, 1H); 5.49 (d, 1H, J=15); 5.14 (d, 1H, J=18); 5.09 (d, 1H, J=10); 4.13 (t, 2H); 2.20 (s, 3H); 1.50 (s, 9H). 13C NMR (125 MHz, CDCl3): 198.6; 153.0; 143.2; 131.8; 117.8; 109.5; 84.0; 47.0; 28.3; 28.1.

### Step 4. tert-Butyl 5-acetyl-2-azabicyclo[2.1.1]hexane-2-carboxylate

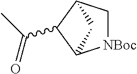

A solution of tert-butyl (E)-allyl(3-oxobut-1-en-1-yl)carbamate (270 g) in MeCN (3240 mL) was subjected to UV-photo reactor. When the reaction was complete, the yellow oil residue (major and minor isomer mixture) was used for next step without further purification. Sample was purified by column to get analytical data. 1H NMR (500 MHz, CDCl₃) δ 4.62-6.78 (bd, 1H); 3.40 (bt, 1H); 3.16 (bs, 1H); 3.06 (bs, 1H); 2.69 (s, 1H); 1.97 (s, 3H); 1.70-1.73 (m, 1H); 1.46 (s, 9H).

### Step 5. tert-Butyl (1R,4R,5S)-5-amino-2-azabicyclo[2.1.1]hexane-2-carboxylate oxalate

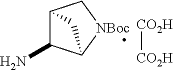

A mixture of tert-butyl 5-acetyl-2-azabicyclo[2.1.1]hexane-2-carboxylate (150 g) in MeCN (1500 mL) was added to sodium hypochlorite (173.5 g) in 30% sodium hydroxide solution at 30-40° C. (1500 mL). The mixture was agitated at 30-40° C. for 30 min. to complete the reaction. The mixture was cooled to 10-15° C. and 6M HCl aq. solution was added to adjust the mixture pH 8-9. The mixture was concentrated under vacuum to remove MeCN at 50-55° C. and methanol (90 mL) was added to the residue. The mixture was cooled to 10-15° C. and 6M HCl was added to adjust the mixture pH 2-3 (solids precipitated out as the pH adjustment) and agitated for additional 2-3 h. The solids were isolated and rinsed with water (300 mL). The wet solids were dried under vacuum at 50-55° C.

Recrystallization: A mixture of the solids in toluene (1500 mL) was heated to 60-70° C. to a solution, (R)-(+)-1-phenylethylamine (80.7 g) was added at 40-70° C. The solution was cooled to 30-35° C. over 90 min. (solids precipitated gradually) and agitated for 1 h. The suspension was cooled to 20-25° C. over 90 min. and agitated for 2 h. The solids were isolated and rinsed with toluene (40 mL). A mixture of the cake and toluene (1200 mL) was heated to 100-105° C. to a solution. The mixture was cooled to 75-85° C. over 90 min. (solids precipitated) and agitated for 1 h. The mixture was cooled to 20-25° C. over 2 h and agitated for 2 h. The solids were isolated and rinsed with toluene (40 mL). The recrystallization process was repeated one more time.

Free base: to a mixture of the wet cake in toluene (225 mL) and water (225 mL) was added 30% aq. NaOH at 10-15° C. to pH 9-10. The mixture was agitated for 30 min. and the organic phase was separated. To the aqueous phase was added 6 M aq. HCl at 10-15° C. to pH 2-3 (solids predicated). The mixture was then cooled to 3-8° C. and agitated for 1 h. The solids were isolated and washed with water (40 mL). The wet cake was dried under vacuum at 50-55° C. to give the desired (1R,4S,5S)-2-(tert-butoxycarbonyl)-2-azabicyclo[2.1.1]hexane-5-carboxylic acid (25 g, 18% yield).

A mixture of the acid (245 g), pyridine (86 g) and ammonium carbonate (111 g) in MeCN (3700 mL) was added (Boc)₂O (310 g) at 15-25° C. The mixture was agitated for 5 h to complete the reaction. The solids were isolated and rinsed with MeCN (250 mL). The filtrate and rinse were combined and concentrated under vacuum at 40-45° C. and azeotroped with heptane. To the residue was added EtOAc (130 mL) and n-heptane (650 mL) at 40-45° C. The mixture was cooled to 10-15° C. (solids precipitated) and agitated for 2 h. The solids were isolated and rinsed with n-heptane (250 mL). The wet cake was dried under vacuum at 50-55° C. to give desired product tert-butyl (1R,4S,5S)-5-carbamoyl-2-azabicyclo[2.1.1]hexane-2-carboxylate quantitatively.

To cooled 15% aq. NaOH (800 mL) at 10-15° C. was added the tert-butyl (1R,4S,5S)-5-carbamoyl-2-azabicyclo[2.1.1]hexane-2-carboxylate (214 g). Sodium hypochlorite (91.2 g) was added at 10-20° C. and the mixture was agitated for 2 h. The mixture was heated to 40-45° C. for 4 h to complete the reaction. The reaction mixture was cooled to 15-20° C. and citric acid was added to adjust pH 5-6. The mixture was basified by addition of sodium hydroxide to pH 14. The basified mixture was extracted with 2-methyltetrahydrofuran (2×1000 mL). The combined organic phase was concentrated under vacuum and the residual was azeotroped with MeCN. The residue was dissolved in (140 mL) and activated charcoal (2 gram) was added. The mixture was agitated at 25-30° C. for 2 h. The mixture was filtered, and the filter bed is rinsed with MeCN (85 mL). The combined filtrate and rinse were added to a solution of oxalic acid (120 g) in MeCN (850 mL) at 40-45° C. The solution was cooled to 3-7° C. and agitated for 1 h. The solids were isolated and rinsed with MeCN (110 mL). The wet cake was dried at 40-50° C. under vacuum to give desired tert-butyl (1R,4R,5S)-5-amino-2-azabicyclo[2.1.1]hexane-2-carboxylate oxalate (248 g, 91% yield) as white solids. HPLC-MS for calc. C₁₀H₁₈N₂O₂: 198.14; Found (M+H): 199.1 ¹H NMR (500 MHz, DMSO-d₆): δ8.44 (s, 3H); 3.34, (m, 1H); 4.24, dt, 1H, J=6.9, 1.7 Hz); 3.20-3.31 (m, 2H); 2.84, (dt, 1H, J=6.5, 3.0); 1.65-1.71 (m, 1H); 1.42 (s, 9H); 1.19 (d, 1H, J=8.1). ¹³C NMR (125 Hz, DMSO-d₆): δ 165.0; 155.8; 79.5; 61.5; 50.6; 44.9; 40.8; 33.8; 28.6.

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A process for preparing a compound of Formula I: 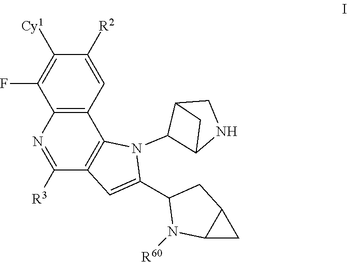
wherein
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
comprising deprotecting a compound of Formula II: 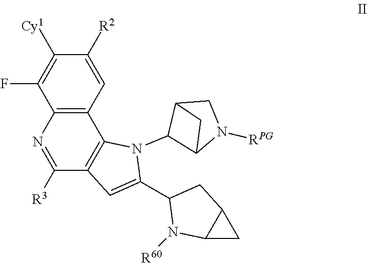
wherein
R^{PG} is a nitrogen protecting group;
to produce the compound of Formula I.

2. The process of claim 1, wherein R^{PG} is a hydrolysable protecting group and the deprotecting comprises hydrolyzing the compound of Formula II.

3. The process of claim 1, wherein the deprotecting comprises reacting the compound of Formula II with an acid.

4. The process of claim 3, wherein the acid is a Lewis acid.

5. The process of claim 3, wherein the acid is HCl.

6. The process of claim 3, wherein the acid is a trialkylsilyl halide.

7. A process for preparing a compound of Formulae II, III, IV, VI, VII, VIII, X-A, XI, XII, XIII, XV, XVI, and XVII, wherein:
(1) the compound prepared is a compound of Formula II: 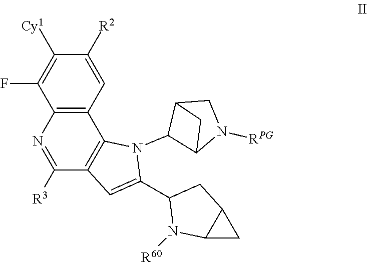
wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
and the process comprises cyclizing a compound of Formula III: 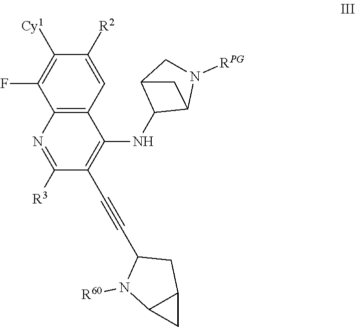
to form the compound of Formula III
(2) the compound prepared is a compound of Formula III: 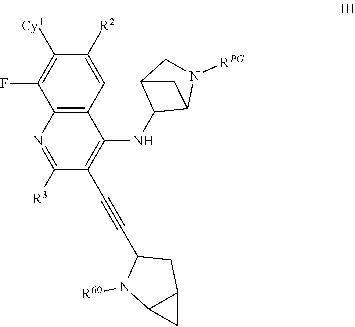
and the process comprises:
coupling a compound of Formula IV: 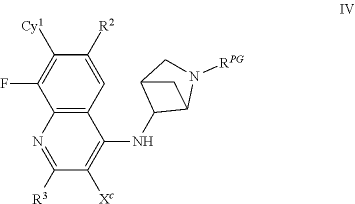
with a compound of Formula V: 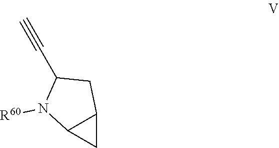
to form the compound of Formula III
wherein
X^{c} is Cl, Br, or I;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
(3) the compound prepared is a compound of Formula IV: 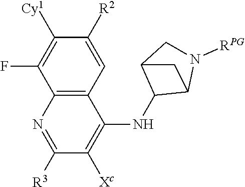
wherein
X^{c} is Cl, Br, or I;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
and the process comprises halogenating a compound of Formula VI: 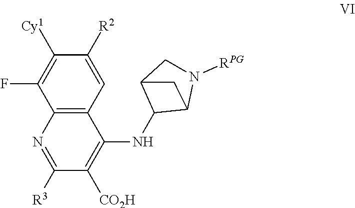
to form the compound of Formula IV;
(4) the compound prepared is a compound of Formula VI: 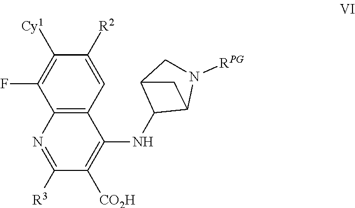
and the process comprises hydrolyzing a compound of Formula VII: 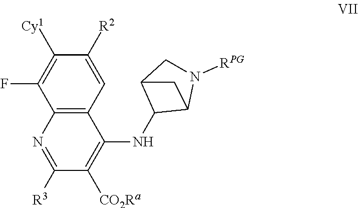
wherein
R^{a} is C₁₋₃ alkyl;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
to form the compound of Formula VI;
(5) the compound prepared is a compound of Formula VII: 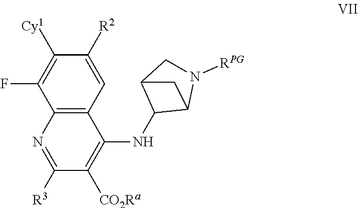
wherein
R^{a} is C₁₋₃ alkyl;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
and the process comprises reacting a compound of Formula VIII: 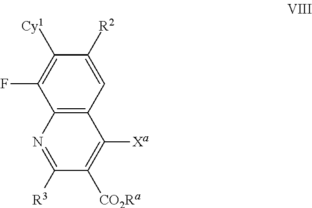
wherein
X^{a} is halo or OH;
with a compound of Formula IX: 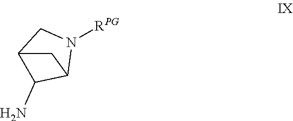
wherein
R^{PG} is a nitrogen protecting group;
to form the compound of Formula VII;
(6) the compound prepared is a compound of Formula VIII: 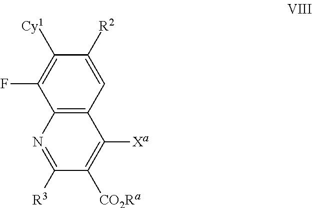
wherein
R^{a} is C₁₋₃ alkyl;
X^{a} is halo or OH;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
and the process comprises reducing a compound of Formula X: 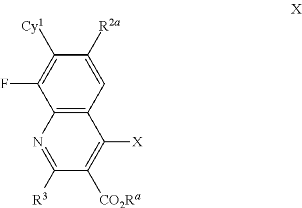
wherein
R^{2a} is selected from C₂₋₄ alkyl and C₂₋₄ alkenyl, both of which are optionally substituted with CN;
to form the compound of Formula VIII;
(7) the compound prepared is a compound of Formula X-A: 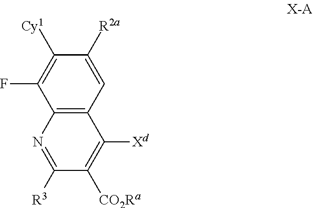
wherein
R^{a} is C₁₋₃ alkyl;
X^{d} is halo;
R^{2a} is selected from C₂₋₄ alkyl and C₂₋₄ alkenyl, both of which are optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
and the process comprises halodehydroxylating a compound of Formula XI: 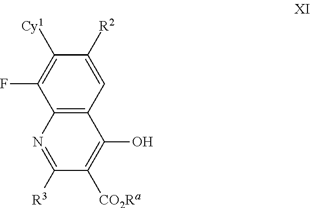
to form the compound of Formula X;
wherein
R² is selected from C₂₋₄ alkyl and C₂₋₄ alkenyl, both of which are optionally substituted with CN;
(8) the compound prepared is a compound of Formula XI: 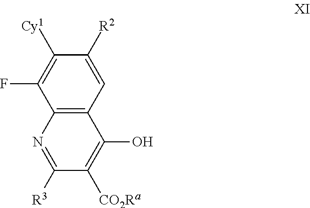
wherein
R^{a} is C₁₋₃ alkyl;
Cy¹ is phenyl; wherein the phenyl is optionally substituted with 1 or 2 substituents independently selected from R¹⁰;
R² is selected from C₂₋₄ alkenyl optionally substituted with CN;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
and the process comprises coupling a compound of Formula XII: 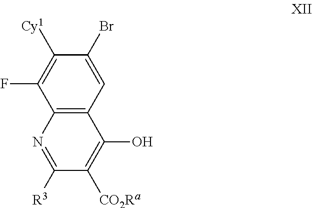
with an alkene form the compound of Formula XI;
(9) the compound prepared is a compound of Formula XII: 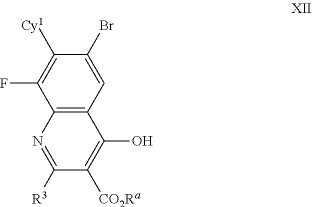
wherein
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
and the process comprises reacting a compound of Formula XIII: 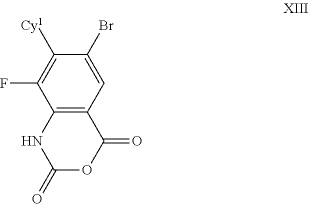
with a compound of Formula XIV: 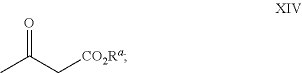
wherein R^{a} is C₁₋₃ alkyl;
to form the compound of Formula XII;
(10) the compound prepared is a compound of Formula XII: 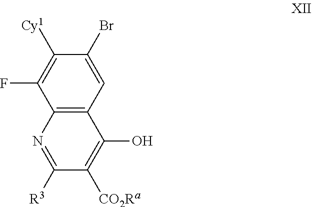
wherein
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
and the process comprises reacting a compound of Formula XIII: 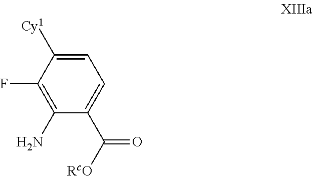
wherein R^{c} is C₁₋₃ alkyl;
with a compound of Formula XIV: 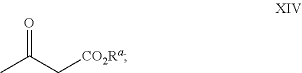
wherein R^{a} is C₁₋₃ alkyl;
to form the compound of Formula XII;
(11) the compound prepared is a compound of Formula XIII: 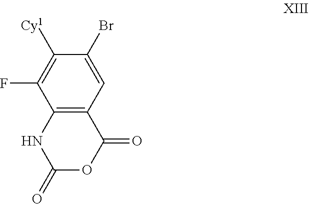
wherein
Cy¹ is phenyl; wherein the phenyl is optionally substituted with 1 or 2 substituents independently selected from R¹⁰; and
each R¹⁰ is independently selected from C₁₋₃ alkyl and halo;
and the process comprises carbonylating a compound of Formula XV: 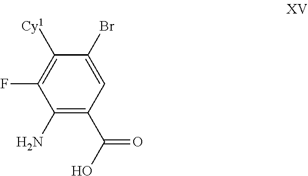
to form the compound of Formula XIII;
(12) the compound prepared is a compound of Formula XV: 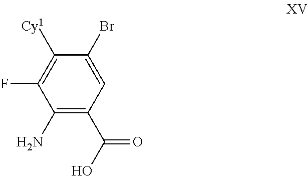
wherein
Cy¹ is phenyl; wherein the phenyl is optionally substituted with 1 or 2 substituents independently selected from R¹⁰;
each R¹⁰ is independently selected from C₁₋₃ alkyl and halo; and
and the process comprises hydrolyzing a compound of Formula XVI: 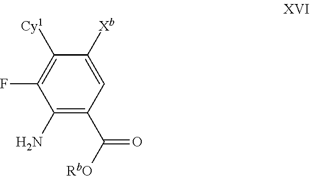
wherein
X^{b} is Cl, Br, or I;
R^{b} is C₁₋₃ alkyl;
to form the compound of Formula XV;
(13) the compound prepared is a compound of Formula XVI: 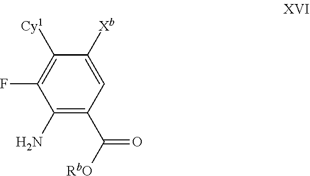
wherein
X^{b} is Cl, Br, or I;
Cy¹ is phenyl; wherein the phenyl is optionally substituted with 1 or 2 substituents independently selected from R¹⁰;
each R¹⁰ is independently selected from C₁₋₃ alkyl and halo; and
R^{b} is C₁₋₃ alkyl;
and the process comprises halogenating a compound of Formula XVII: 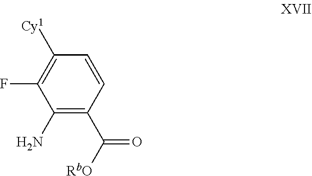
wherein
to form the compound of Formula XVI;
(14) the compound prepared is a compound of Formula XVII: 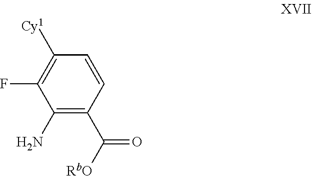
wherein
R^{b} is C₁₋₃ alkyl;
Cy¹ is phenyl; wherein the phenyl is optionally substituted with 1 or 2 substituents independently selected from R¹⁰;
and the process comprises coupling a compound of Formula XVIII: 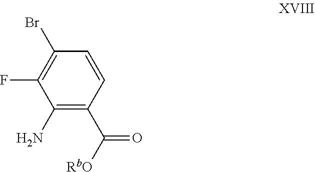
with a compound of Formula XIX: 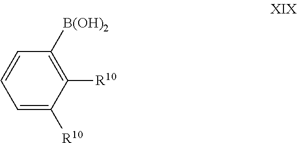
or an ester thereof;
wherein
each R¹⁰ is independently selected from C₁₋₃ alkyl and halo;
in the presence of a palladium catalyst to form the compound of Formula XVII; or
(15) the compound prepared is a compound of Formula XVII: 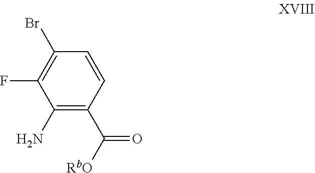
wherein
R^{b} is C₁₋₃ alkyl;
and the process comprises esterifying a compound of Formula XX: 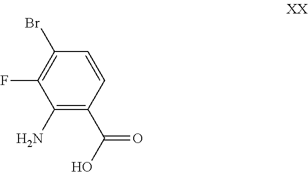 [SMILES: Nc1c(C(=O)O)ccc(Br)c1F]
to form the compound of Formula XVIII.

8-73. (canceled)

74. The process of claim 1, wherein R² is CH₂CH₂CN;
Cy¹ is 2,3-dichlorophenyl;
R³ is methyl;
R⁶⁰ is C(O)R^{b60};
R^{b60} is cyclopropyl; and
R^{PG} is tert-butyloxycarbonyl.

75-79. (canceled)

80. The process of claim 1, wherein the compound of Formula I is 3-(1-(2-azabicyclo[2.1.1]hexan-5-yl)-2-(2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt, hydrate, of solvate thereof.

81. The process of claim 1, wherein the compound of Formula I is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

82. The process of claim 1, wherein the compound of Formula I is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

83. (canceled)

84. The process of claim 1, wherein the compound of Formula II is tert-butyl 5-(8-(2-cyanoethyl)-2-(2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

85. The process of claim 1, wherein the compound of Formula II is tert-butyl (1R,4R,5S)-5-(8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

86. The process of claim 1, wherein the compound of Formula II is tert-butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

87. (canceled)

88. The process of claim 7, wherein the compound of Formula III is tert-butyl 5-((6-(2-cyanoethyl)-3-((2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate;
the compound of Formula IV is tert-butyl 5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula V is cyclopropyl(3-ethynyl-2-azabicyclo[3.1.0]hexan-2-yl)methanone;
the compound of Formula VI is 4-((2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl 5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate; and
the compound of Formula VIII is ethyl 4-chloro-6-(2-cyanoethyl)-7-(2,3-dichlorophenyl)-8-fluoro-2-methylquinoline-3-carboxylate or ethyl 6-(2-cyanoethyl)-7-(2,3-dichlorophenyl)-8-fluoro-4-hydroxy-2-methylquinoline-3-carboxylate.

89. The process of claim 7, wherein the compound of Formula III is tert-butyl (1R,4R,5S)-5-((6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate;
the compound of Formula IV is tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate; and
the compound of Formula V is cyclopropyl((1R,3R,5R)-3-ethynyl-2-azabicyclo[3.1.0]hexan-2-yl)methanone;
the compound of Formula VI is 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid; and
the compound of Formula VII is tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate.

90. The process of claim 7, wherein the compound of Formula III is tert-butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate;
the compound of Formula IV is tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula VI is (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate; and
the compound of Formula VIII is ethyl (Rₐ)-4-chloro-6-(2-cyanoethyl)-7-(2,3-dichlorophenyl)-8-fluoro-2-methylquinoline-3-carboxylate or ethyl (Rₐ)-6-(2-cyanoethyl)-7-(2,3-dichlorophenyl)-8-fluoro-4-hydroxy-2-methylquinoline-3-carboxylate.

91-122. (canceled)

123. A compound of Formula II, III, IV, VI, VII, VIII, X, XI, XII, XIII, XV, XVI, or XVII, wherein:
(1) the compound is a compound of Formula II: 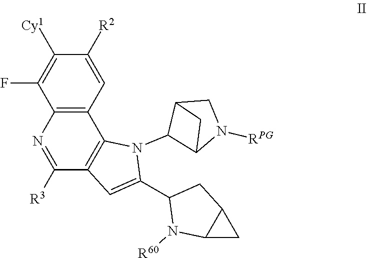
wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
(2) the compound is a compound of Formula III: 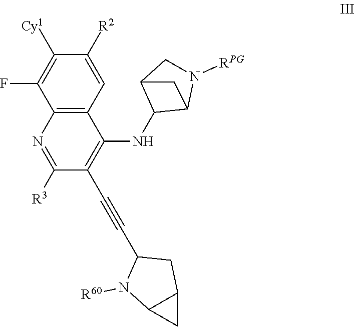
wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo;
each R³⁰ is independently selected from C₁₋₃ alkyl, halo, and D;
R⁶⁰ is selected from C₁₋₃ alkyl, halo, and C(O)R^{b60}; and
R^{b60} is selected from H, C₁₋₃ alkyl, C₁₋₃ haloalkyl, and C₃₋₆ cycloalkyl;
(3) the compound is a compound of Formula IV: 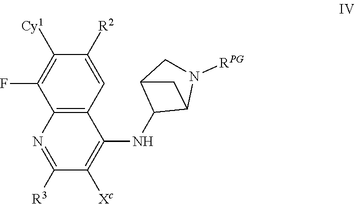
wherein
X^{c} is Cl, Br, or I;
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(4) the compound is a compound of Formula VI: 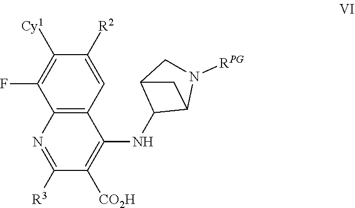
wherein
R^{PG} is a nitrogen protecting group;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(5) the compound is a compound of Formula VII: 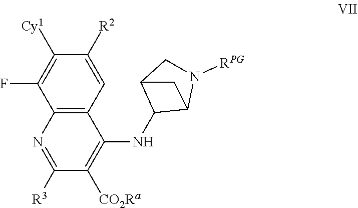
wherein
R^{a} is C₁₋₃ alkyl;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(6) the compound is a compound of Formula VIII: 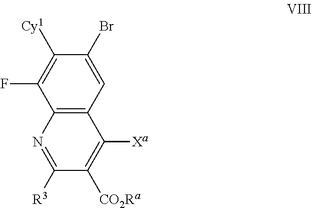
wherein
X^{a} is halo or OH;
R^{a} is C₁₋₃ alkyl;
R² is selected from C₂₋₄ alkyl optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(7) the compound is a compound of Formula X: 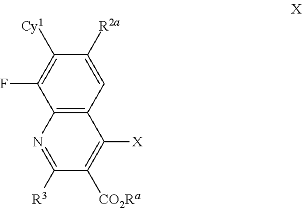
wherein
R^{a} is C₁₋₃ alkyl;
X is halo;
R^{2a} is selected from C₂₋₄ alkyl and C₂₋₄ alkenyl, both of which are optionally substituted with CN;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(8) the compound is a compound of Formula XI: 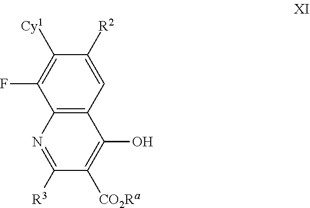
wherein
R^{a} is C₁₋₃ alkyl;
Cy¹ is phenyl; wherein the phenyl is optionally substituted with 1 or 2 substituents independently selected from R¹⁰;
R² is selected from C₂₋₄ alkenyl optionally substituted with CN;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(9) the compound is a compound of Formula XII: 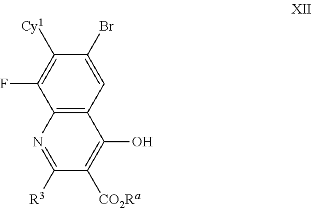
wherein
R^{a} is C₁₋₃ alkyl;
Cy¹ is phenyl; wherein the phenyl is optionally substituted with 1 or 2 substituents independently selected from R¹⁰;
R³ is C₁₋₃ alkyl optionally substituted with 1, 2, or 3 substituents independently selected from R³⁰;
each R¹⁰ is independently selected from C₁₋₃ alkyl and halo; and
each R³⁰ is independently selected from C₁₋₃ alkyl, halo, and D;
(10) the compound is a compound of Formula XIII: 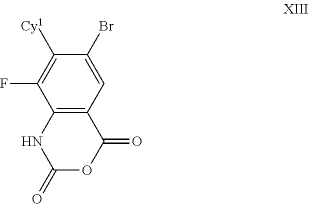
wherein
Cy¹ is phenyl; wherein the phenyl is optionally substituted with 1 or 2 substituents independently selected from R¹⁰; and
each R¹⁰ is independently selected from C₁₋₃ alkyl and halo;
(11) the compound is a compound of Formula XV: 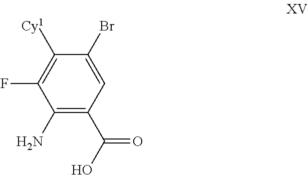
wherein
Cy¹ is phenyl; wherein the phenyl is optionally substituted with 1 or 2 substituents independently selected from R¹⁰; and
each R¹⁰ is independently selected from C₁₋₃ alkyl and halo;
(12) the compound is a compound of Formula XVI: 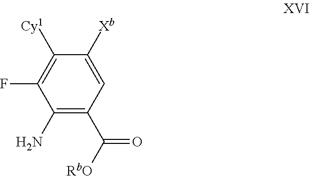
wherein
X^{b} is Cl, Br, or I;
R^{b} is C₁₋₃ alkyl;
Cy¹ is phenyl; wherein the phenyl is optionally substituted with 1 or 2 substituents independently selected from R¹⁰; and
each R¹⁰ is independently selected from C₁₋₃ alkyl and halo; or
(13) the compound is a compound of Formula XVII: 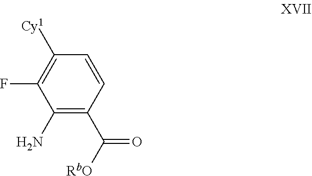
wherein
Cy¹ is phenyl; wherein the phenyl is optionally substituted with 1 or 2 substituents independently selected from R¹⁰;
each R¹⁰ is independently selected from C₁₋₃ alkyl and halo; and
R^{b} is C₁₋₃ alkyl.

124. The compound of claim 123, wherein R² is CH₂CH₂CN;
Cy¹ is 2,3-dichlorophenyl;
R³ is methyl;
R⁶⁰ is C(O)R^{b60};
R^{b60} is cyclopropyl; and
R^{PG} is tert-butyloxycarbonyl.

125-129. (canceled)

130. The compound of claim 123, wherein the compound of Formula II is tert-butyl 5-(8-(2-cyanoethyl)-2-(2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

131. The compound of claim 123, wherein the compound of Formula II is tert-butyl (1R,4R,5S)-5-(8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

132. The compound of claim 123, wherein the compound of Formula II is tert-butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

133-140. (canceled)

141. The compound of claim 123, wherein the compound of Formula III is tert-butyl 5-((6-(2-cyanoethyl)-3-((2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate;
the compound of Formula IV is tert-butyl 5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula VI is 4-((2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl 5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate; and
the compound of Formula VIII is ethyl 4-chloro-6-(2-cyanoethyl)-7-(2,3-dichlorophenyl)-8-fluoro-2-methylquinoline-3-carboxylate.

142. The compound of claim 123, wherein the compound of Formula III is tert-butyl (1R,4R,5S)-5-((6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate;
the compound of Formula IV is tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula VI is 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid; and
the compound of Formula VII is tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate.

143. The compound of claim 123, wherein the compound of Formula III is tert-butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate;
the compound of Formula IV is tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate;
the compound of Formula VI is (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid;
the compound of Formula VII is tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate; and
the compound of Formula VIII is ethyl (Rₐ)-4-chloro-6-(2-cyanoethyl)-7-(2,3-dichlorophenyl)-8-fluoro-2-methylquinoline-3-carboxylate.

144-213. (canceled)
